(19) United States
(12) Patent Application Publication
(10) Pub. No.: US 2013/0245582 A1
(43) Pub. Date: Sep. 19, 2013

(54) WOUND DRESSING FOR THE ABDOMINAL REGION

(71) Applicants: Pierre Croizat, Herbrechtingen (DE); Axel Eckstein, Heidenheim (DE); Cornelia Wolf, Herbrechtingen (DE)

(72) Inventors: Pierre Croizat, Herbrechtingen (DE); Axel Eckstein, Heidenheim (DE); Cornelia Wolf, Herbrechtingen (DE)

(73) Assignee: Paul Hartmann AG, Heidenheim (DE)

(21) Appl. No.: 13/605,311

(22) Filed: Sep. 6, 2012

Related U.S. Application Data
(60) Provisional application No. 61/533,443, filed on Sep. 12, 2011.

(30) Foreign Application Priority Data
Sep. 9, 2011 (EP) EP 11007378.0

(51) Int. Cl. A61F 13/14; A61F 13/00

(52) U.S. Cl. CPC A61F 13/148; A61F 13/00987
USPC 604/319; 29/428

(57) ABSTRACT

The present invention relates to a wound dressing for use in negative-pressure wound therapy, in particular for wounds in the abdominal region. The wound dressing comprises a first flexible film with a first and a second side, with the first side being provided for application on the wound bed, more particularly on exposed internal organs or on the greater omentum, and with the first film furthermore having a multiplicity of openings distributed across the area. Moreover, the wound dressing comprises at least three conduit sections, applied to the second side of the first film, made of a flexible elastomeric material with a thickness (H) of at most 20 mm, with each of the conduit sections having at least one continuous cavity, and with each of the conduit sections having a flat design. Furthermore, the wound dressing is distinguished by virtue of the fact that, with the exception of the openings situated at the ends, each of the conduit sections has no further openings.

### CROSS REFERENCE TO RELATED APPLICATIONS

This application claims priority to U.S. Provisional Application No. 61/533,443 filed Sep. 12, 2012, which is herein incorporated by reference.

### BACKGROUND

1. Field of the Invention

The present invention relates to a wound dressing and to a device for use in negative-pressure wound therapy, in particular for wounds in the abdominal region, and also to methods for producing the device.

2. Background of the Art

Devices for negative-pressure wound therapy and bandages as components is of such devices are known from the prior art.

Thus, for example, WO1993/009727 describes a device for promoting wound healing by the application of negative pressure to the region of skin having the wound and surrounding the wound. The device as per WO1993/009727 comprises a vacuum apparatus for producing the negative pressure, an airtight cover of the wound referred to as sealing apparatus, which is functionally connected to the vacuum apparatus, and also a wound dressing referred to as screen apparatus for positioning on the wound within the sealing apparatus. The screen apparatus is a porous polymer foam, for example polyester foam. According to the description of WO1993/009727, the wound healing of different types of wounds, such as burn wounds, pressure sores or lacerations, can be accelerated by the application of negative-pressure therapy.

Here, the term “negative pressure” refers to an air pressure within the wound bandage that is reduced compared to the ambient air pressure (atmospheric air pressure). “Within the wound bandage” is understood to mean the interspace (wound space) formed by the airtight cover material and the body tissue in the wound region. “Negative pressure” is often also referred to as “low pressure”. In the context of the invention, the pressure difference between the air pressure within the wound bandage and the ambient air pressure is specified in mm Hg (millimeters of mercury) because this is conventional in the field of negative-pressure therapy. 1 mm Hg corresponds to one Torr or 133.322 Pa (pascal). In the context of the invention, the negative pressure, i.e. the pressure difference between the air pressure within the wound bandage and the ambient air pressure, is specified as a positive number in mm Hg.

Particularly large-area wounds can be created in the abdominal region, either as a result of injury or as a result of surgical interventions. By way of example, surgical interventions in the abdominal region are undertaken in the case of surgical treatment of acute and life-threatening diseases of the abdominal cavity. Within the is scope of post-operative care of such surgical interventions, it may also be necessary to cover the open abdominal region only temporarily by means of a temporary wound closure.

In the case of an abdominal wound, exposed internal organs or the greater omentum (also referred to as omentum majus or great omentum) form a wound bed, i.e. a body surface situated within a wound edge. When tending an abdominal wound, a layer of the wound dressing oriented toward the wound (said layer is referred to as wound contact layer below) is directly applied to exposed internal organs or to the greater omentum. The wound contact layer lying on the exposed internal organs or on the greater omentum also serves as an organ screening layer during the negative-pressure treatment of an abdominal wound and should prevent organs or the greater omentum from inadvertently adhering to the abdominal wall. The edge region of the wound dressing is usually inserted into the interspace formed by the abdominal wall and internal organs.

During the treatment of a wound on the open abdomen, it may be necessary to drain a very large amount of liquid, for example up to 5 l within 48 hours. By way of example, very large amounts of liquid to be drained can be created during the surgical treatment of a bowel obstruction (ileus) or an inflammation of the peritoneum (peritonitis).

WO01/85248 has disclosed a bandage for temporarily covering wounds from accidents or surgical interventions, more particularly abdominal wounds. The bandage is provided for use in negative-pressure therapy. WO01/85248 proposes to cover the wound bed with a film provided with holes. A porous foam is applied to the film which constitutes the wound contact layer. On the side that faces away from the wound, the bandage comprises a cover film which is impermeable to liquids and has an adhesive edge for sealing the wound region in an airtight fashion. Furthermore, provision is made for connection means which extend through the cover film as far as the porous foam in order to be able to connect the wound space to a negative-pressure source. During operation, wound exudate can be removed from the wound space by virtue of the liquid first of all reaching the porous foam through the openings is in the perforated film and furthermore reaching the connection means via the foam, said connection means being in direct contact with the porous foam.

WO01/85248 is directed, in particular, to a wound bandage that can be replaced without damage to or traumatization of the wound. Such damage when the bandage is changed can occur if the wound bandage adheres during the therapy to the tissue situated therebelow or if tissue grows into the tissue, in particular if fibrous tissue grows into the foam portion of the wound bandage. WO01/85248 wishes to reduce the undesired growth of tissue into the bandage by virtue of reducing the direct contact between porous material and wound bed. In this respect, WO01/85248 proposes that the area of the openings in the film provided with holes is less than 10% of the effective area of the film. Ideally, the open area should be less than 1 or 2% of the area of the film. According to WO01/85248, the openings in the film can be present in the form of slits, which further reduces the contact between foam and wound bed.

WO2010/051068 describes a wound dressing for the open abdomen, the former comprising a multiplicity of enveloped strand-like pressure distribution elements which are functionally coupled by a central connection element. A further pressure distribution element is applied to the side of the central connection element that faces away from the wound. By way of example, the pressure distribution elements can be an open-cell foam. The wound dressing can be cut to size.

US2009/0099519 describes an abdominal bandage for negative-pressure therapy, which comprises an enveloped cushion and a heater element for controlling the temperature.

WO2010/124844 discloses a wound dressing which, in particular, is provided for use on the open abdomen. The wound dressing comprises two web-like elements, which form a drainage space situated between their inner delimiting surfaces. At least one of the web-like elements can have openings, with provision in particular being made for canal-shaped openings that open into the drainage space and can promote capillary action.

The patent application DE102010052336.4 (not yet published at the time of the present application) by the applicant of the present patent application describes a bandage for use in negative-pressure wound therapy, in particular for wounds in the abdominal region, comprising a flexible film as wound contact layer and at least one conduit applied to the film, with both the conduit and the film having a multiplicity of openings such that a fluid connection can be established between the conduit and the wound space. Here, in addition to the openings situated at the ends, the conduit applied to the film has a multiplicity of lateral openings which render it possible to take up wound fluid into the continuous cavity.

In the case of draining large-area and strongly exuding wounds in the abdominal region, there is the need, in particular, to ensure a constant and uniform distribution of the drainage capacity over the whole area of the wound. In particular, a drainage capacity which reduces toward the edges of the wound dressing should be avoided because there may otherwise be an undesired collection of liquids in the peripheral region of the abdomen.

When applying an abdominal wound dressing to exposed internal organs or the greater omentum, the user, e.g. a surgeon, must take care that there is even application of the bandage on the wound bed. In particular, the user may find it difficult to apply the encircling edge region of the wound dressing between abdominal wall and internal organs.

### SUMMARY OF THE INVENTION

It is an object of the present invention to provide an improved abdominal wound dressing for negative-pressure therapy. In particular, it is an object of the present invention to provide an abdominal wound dressing for negative-pressure therapy, with increased drainage capacity and improved user friendliness. Here, the abdominal wound dressing should comprise a drainage capacity which ensures uniform drainage of wound exudate over the whole area of the wound, including the peripheral cavities of the abdomen, during the whole duration of the negative-pressure therapy. It should be easy to apply the wound dressing, match it to the conditions of the individual wound well and replace it without adhesion or growth into the bed.

According to the invention, this object is achieved by a wound dressing for use in negative-pressure wound therapy, in particular for treatment of abdominal wounds, according to claim 1. The wound dressing comprises a first flexible film with a first and a second side, with the first side being provided for application on the wound bed, more particularly on exposed internal organs or on the greater omentum, and with the first film furthermore having a multiplicity of openings distributed across the area. Moreover, the wound dressing comprises at least three conduit sections, applied to the second side of the first film, made of a flexible elastomeric material with a thickness (H) of at most 20 mm, with each of the conduit sections having at least one continuous cavity, and with each of the conduit sections having a flat design. Furthermore, the wound dressing is distinguished by virtue of the fact that, with the exception of the openings situated at the ends, each of the conduit sections has no further openings. During the application in negative-pressure wound therapy, the wound dressing can be connected in a fluid-conducting fashion to a negative-pressure source such that fluid-communication can be established between the negative-pressure source and the wound space.

The thickness (H) is the maximum extent of a conduit section applied to a first film, in a dimension running perpendicular to the plane of the first film.

The cover film is usually attached to the region of the skin surrounding the wound in a sealing fashion. In this context, a fluid is understood to mean both liquids, e.g. wound fluid, blood, intestinal fluid or rinsing fluid, and also gases, e.g. air or carbon dioxide.

Compared to wound dressings known from the prior art, the wound dressing according to the invention provides further cavities in the form of at least three conduit sections which have a flexible and flat design, for supporting the drainage of is wound exudate. As a result of this it is possible to increase the drainage capacity of the wound dressing substantially, particularly in the peripheral regions, and furthermore stabilize it during the duration of the application. The wound dressing according to the invention ensures continuous and disturbance-free drainage of wound fluid over the application duration of the device, for example over a period of 24 to 72 hours. Here, continuous and disturbance-free drainage of wound fluid from the peripheral regions of the wound is more particularly ensured via the lumina of the at least three conduit sections because the latter are less susceptible to blockages, for example compared to a foam ply, as a result of their open cross-sectional area.

The conduit sections with a flexible and flat design ensure both drainage of wound fluid from the peripheral regions of the abdomen and also continuous and disturbance-free application of negative pressure (i.e. setting an air pressure that is reduced compared to the ambient air pressure) in the wound space for the duration of the therapeutic application. A pressure drop in the peripheral region of the abdomen, i.e. a reducing negative pressure in the edge region of the wound space, is avoided.

The wound dressing according to the invention can be matched without restrictions to the shape and size of the wound by the user, i.e. by the medical practitioner or by the medical staff, for example by simple cutting to size by means of sterile scissors. By contrast, to the extent that it is desired to provide the product matched to predefined wound shapes and wound dimensions already during production, there are likewise no restrictions in this respect as a result of the design of the wound dressing according to the invention.

The wound dressing according to the invention comprises a first flexible film with a first and a second side. During use, the first side of the flexible film is placed into direct contact with the wound bed, i.e. more particularly onto the internal organs or the greater omentum exposed during surgery or as a result of injury. It is therefore essential for the first flexible film to consist of a material which does not adhere to or grow together with the exposed internal organs, the greater omentum or the is abdominal wall for the duration of the application. The material should have atraumatic properties.

Suitable materials for the first flexible film comprise thermoplastic films, more particularly films made of ethylene-vinyl acetate (abbreviated EVA below), polyurethane (abbreviated PU below), polyethylene (abbreviated PE below), polyethylene terephthalate (abbreviated PET below), PTFE (referred to as polytetrafluoroethylene below), polyvinyl chloride (abbreviated PVC below), thermoplastic elastomers (abbreviated TPE below), polyorganosiloxane (shortened to silicone below) or a mixture thereof. In this context, the label TPE comprises thermoplastic elastomers based on olefins (TPO), cross-linked thermoplastic elastomers based on olefins (TPV), thermoplastic elastomers based on urethane (TPU), thermoplastic polyester elastomers or thermoplastic copolyesters (TPC), styrene block copolymers (TPS) and thermoplastic copolyamides (TPA). The thermoplastic film is preferably a PE film.

The mass per unit area of the first film should be at least 30 g/m² and at most 150 g/m², preferably at least 45 g/m² and at most 95 g/m² and more particularly at least 55 g/m² and at most 65 g/m².

According to a particularly preferred embodiment, the first flexible film is a support film-free PE film with a mass per unit area of 55 g/m² to 65 g/m², with the mass per unit area being determined pursuant to the norm EN ISO 2286-2, for example the products “Folie MEDIFOL® 3D, Type 44600” or “Folie MEDIFOL® 3D T16, Type 44601 T16” by rkw Prolife (Wasserburg, Germany).

The first flexible film has a multiplicity of openings which enable wound fluid to pass therethrough. The openings can be holes or slits. In this context, a hole refers to an opening which, in a plan view and in the non-stretched state, has an open area. In this context, a slit refers to an opening which, in a plan view and in the non-stretched state, has no open area. The desired permeability of the film in respect of wound fluid can be set in suitable fashion by the shape and dimension of the openings. Round, oval, polygonal or e.g. star-shaped holes are feasible. By way of example, the slits is can be elongate or cross-shaped slits.

The openings can be present distributed in a regular, i.e. in regularly repeating patterns, or random fashion across the area of the first film. In the process, it is also possible for only a first portion of the first flexible film to have a multiplicity of openings distributed across the area while a further portion of the first flexible film has no openings. Thus, for example, it can be advantageous for a central portion of the first film to have a multiplicity of openings while a peripheral portion of the film has no openings. A reverse arrangement is likewise possible.

Should the openings be slits, these should respectively have a length of at least 1 mm and at most 30 mm, preferably at least 2 mm and at most 20 mm, and more particularly at least 5 mm and at most 10 mm. In this case it was found that a film which has slits with a length of at least 5 mm and at most 10 mm, with between 10 and 90 of such slits being introduced on a film area of 100 cm², has particularly advantageous properties in respect of stability and at the same time has sufficient fluid permeability.

The first flexible film preferably has a multiplicity of openings, with the openings being holes. The diameter of the holes can be adjusted in a suitable manner in respect of the desired fluid permeability, for example in a range between 0.1 mm and 5 mm in the case of circular holes. Ranges between at least 0.2 mm and at most 0.4 mm are particularly preferred in this case in respect of an expedient combination of fluid permeability and atraumatic properties of the film. In this case, holes with a diameter of 0.3 mm have particularly expedient properties.

If the openings in the first film are holes, the sum of the open area of the holes should moreover be at least 0.5%, but preferably at least 10%, of the areal extent of the film in order to ensure sufficient permeability of the film for wound fluid. In the process, an open area of 25% of the film should, where possible, not be exceeded because this could otherwise have a negative effect on the stability of the film. Hence it is preferable for the sum of the open area of the holes present in the film to be at is least 0.5% and at most 25% of the areal extent, preferably at least 12% and at most 23% of the areal extent.

Fluid permeability, stability and the tendency of tissue to grow into the film are influenced by the open area of the film.

According to a preferred embodiment of the invention, the open area of the holes present in the first film is at least 13% and at most 15% of the areal extent of the film. Such a film is found to be advantageous, particularly in respect of fluid permeability, atraumatic properties and stability. In this embodiment, the number of the openings present in the first film per unit area should be at least 150 per cm² and at most 190 per cm², more particularly at least 165 per cm² and at most 171 per cm².

According to a further preferred embodiment of the invention, the open area of the holes present in the first film is at least 20% and at most 22% of the areal extent of the film. Such a film has a high fluid permeability and flexibility or softness. In this further embodiment, the number of the openings present in the first film per unit area should be at least 260 per cm² and at most 300 per cm², more particularly at least 275 per cm² and at most 285 per cm².

It is also feasible for the first film to have a multiplicity of openings distributed across the area, with the openings being a combination of holes and slits. By way of example, this can be embodied such that a specific surface region of the first film has holes and another surface region of the first film has slits.

The invention comprises further variants, not specifically mentioned here, in respect of shape, dimension, number and arrangement of the openings in the first flexible film.

The openings are usually introduced into the first film prior to attaching the at least three conduit sections, or a film which already has openings introduced by the producer is used as initial material. However, it is also possible to create the openings only after the application of the conduit sections.

According to a particularly preferred embodiment of the invention, the openings present in the first film are produced by perforation or stamping the film such that openings which, to the greatest possible extent, have a conical or cylindrical shape are created, which have a three-dimensional structure such that, as a result of this, the film has a smooth side and a roughened side which lies opposite to the smooth side. By way of example, openings with a three-dimensional structure can be introduced by means of a stamping roller. Alternatively, openings with a three-dimensional structure can already be created during the production of the film, for example by guiding the extruded, still molten film over a rotating, low-pressure perforated roller.

Particular advantages when using a first film with such three-dimensionally structured openings for the wound bandage according to the invention emerge in particular if the first side of the first film, provided for application on the wound bed, is formed by the smooth side of the film and the second side of the first film is formed by the roughened side of the film. The smooth side of the first film, provided for application to the wound bed, has a low tendency to adhere to the wound bed. Secondly, the three-dimensionally structured openings bring about a spacing of the wound bed to further plies of the bandage, e.g. to a foam ply, if such additional bandage layers are present. A spacing from further plies can reduce undesirable growing of fibrous tissue into further bandage plies.

Films that have conically or cylindrically shaped openings are commercially available, e.g. the aforementioned films with the names “Folie MEDIFOL® 3D, Type 44600” or “Folie MEDIFOL® 3D T16, Type 44601 T16” (produced by rkw ProLife, Wasserburg, Germany).

At least three conduit sections made of a flexible elastomeric material with a thickness (H) of at most 20 mm are applied to the second side of the first film, with each of the conduit sections having at least one continuous cavity, and with each of the conduit sections having a flat design. Here, with the exception of the openings situated at the ends, each of the conduit sections has no further openings.

The composite of first film and conduit sections should have sufficient flexibility and softness overall such that the wound dressing can nestle closely against the wound bed. Flexibility and softness of the composite of first film and conduit sections are determined by the respective properties of the individual components—the film and conduit. Therefore it is essential that both the first film and the conduit sections applied to the first film consist of a flexible elastomeric material. According to a preferred embodiment, each of the conduit sections should therefore be made of a material which has a Shore A hardness of at most 70 (determined pursuant to DIN 53505 from August 2000, to be precise at 23° C. on a plate-shaped level and flat sample body with a thickness of 6 mm as described in the norm). In a particularly advantageous fashion even softer materials are used for a conduit section, i.e. materials with a Shore A hardness of less than 65, in particular with a Shore A hardness of less than 62, particularly preferably with a Shore A hardness of 60. Flexibility and softness of first film and conduit section moreover simplify the option of cutting to size for the purpose of matching the wound dressing to the wound shape.

The at least three conduit sections are preferably a section of a single-lumen or multi-lumen drainage tube, with the drainage tube for example comprising EVA, PU, PVC, PE, PET, PTFE, TPE, silicone or a mixture thereof. A conduit section suitable within the scope of the invention can be obtained by cutting a longer drainage tube to length.

Furthermore, it is essential in respect of the flexibility of the composite of first film and conduit sections that each of the at least three conduit sections has a thickness (H) of at most 20 mm, preferably of less than 15 mm, more particularly of less than 7 mm.

The at least three conduit sections applied to the first film have a flat design, i.e. they preferably have an overall flat shape in respect of their outer circumference. In the context of the invention, an overall flat shape of a conduit section is understood to mean that a conduit section has a ratio between its transverse extent or width B (maximum extent of the cross section of the conduit section in a dimension running perpendicular to the plane of the first film) and thickness or height H (maximum extent of the conduit section applied to a first film in a dimension running perpendicular to the plane of the first film) of more than 1.25, preferably of more than 1.5, more particularly of more than 3, with the thickness (H) being at most 20 mm, preferably less than 15 mm, more particularly less than 7 mm. The overall flat shape of the at least three conduit sections is advantageous for ensuring a connection to the first film which is stable during use and optionally non-detachable. Furthermore, the overall flat shape of the conduit sections is found to be advantageous when applying the wound dressing and when wearing the wound dressing during the negative-pressure therapy.

According to one advantageous embodiment of the invention, the width B of the conduit section is between 8 mm and 40 mm, more particularly between 15 and 35 mm, in this case. According to a particularly preferred embodiment of the invention, the conduit section has a width B of between 18 and 22 mm and a thickness H of 5 to 6 mm, with the conduit section being made of a material with a Shore A hardness of 60.

According to the invention, the at least three conduit sections are applied to the second side of the first film. In the context of the present invention, “applied” is understood to mean that a) a conduit section is connected to the second side of the first film in a non-detachable and areal fashion and/or that b) a conduit section is held on the film on the second side of the first film by attachment means.

In the case of the first option a), a conduit section should more particularly be connected in a non-detachable and areal fashion to the second side of the first film with at least 10%, preferably with at least 25%, more particularly with at least 50%, of its area projected perpendicularly onto the first film. In this context, “connected in a non-detachable fashion” is understood to mean that a conduit section does not detach from the first film during normal therapeutic use of the wound dressing. A non-detachable connection of the conduit sections to the first film within the aforementioned meaning can for example be achieved by adhesive bonding, welding is or by the use of a double-sided adhesive tape. The conduit section particularly preferably is a drainage tube with an overall substantially rectangular external circumference, in which at least 50% of its area projected perpendicularly onto the first film can be connected to the second side of the first film in a non-detachable and areal fashion.

In the case of the second option b), a conduit section should be held on the first film by attachment means. Here, attachment means are understood to mean a multiplicity of attachment means known to a person skilled in the art, for example adhesive strips or fasteners. The attachment means can also be a second film, which is connected to the second side of the first film and can hold a conduit section in a pocket-like manner between first and second film.

A conduit section has a lumen in the form of one or more continuous cavities in order to ensure drainage of wound fluid to the negative-pressure source and to ensure negative pressure in the wound space. Accordingly, a conduit section can comprise either only one cavity or else a plurality of cavities for forming the lumen.

If a conduit section in each case has a plurality of cavities, the individual cavities of the conduit section can be interconnected via openings to form a continuous lumen. However, it is also feasible that a conduit section has a plurality of continuous cavities which are not interconnected. Here the overall open cross-sectional area of the lumen should be at least 1 mm², preferably at least 5 mm², in order to provide sufficient through-flow capacity for draining wound fluid. According to a particularly preferred embodiment, the lumen of each individual conduit section should have a cross-sectional area of 10 to 50 mm², preferably 20 to 35 mm², overall. In this context, the “overall open cross-sectional area of the lumen” is understood to mean the sum of the open cross-sectional areas of the individual cavities of an individual conduit section, with the conduit section being in a non-compressed state.

A conduit section that can be used within the scope of the invention preferably has two or three continuous cavities which are more particularly interconnected in a is fluid-conducting fashion.

According to a further particularly preferred embodiment, each of the at least three conduit sections only has a single continuous cavity.

In this case it is essential that each of the conduit sections has sufficient negative-pressure stability. In the context of the invention, a sufficient negative-pressure stability of the conduit section is understood to mean that the conduit section does not completely collapse if negative pressure is applied such that a fluid flow through the conduit section is possible. In particular, a sufficient negative-pressure stability is understood to mean that the conduit section retains an open cross section of at least 1 mm² in the case of a negative pressure of typically 125 mm Hg, which is used in negative-pressure therapy. Undesired compression of the conduit in the case of negative pressure can be prevented in different manners known to a person skilled in the art, for example by a suitable cross-sectional geometry and/or by selecting suitable materials. Thus, for example, asymmetrically arranged internal wall ribs can be provided in the conduit section, the former having an advantageous effect on the negative-pressure stability of the conduit section. Conduits with a sufficient negative-pressure stability suitable according to the invention are also commercially available as finished product with a suitable quality for medical applications. Such conduits are usually referred to as drainage tubes.

According to a particularly advantageous embodiment, the conduit section has a coating of or impregnation with a substance with an anticoagulating effect (for example Heparin or another substance with anticoagulating effect usually used for coating medical tubes or tubules). Such an anticoagulating coating can prevent the conduit section from becoming blocked by blood clots such that a uniform high throughput of wound exudate remains ensured over a relatively long period of application. Medical drainage tubes coated with an anticoagulant are commercially available as finished product, for example from Axiom Medical (Burstadt, Germany).

The at least three conduit sections applied to the first film are situated on the second side of the first film which faces away from the wound. The at least three is conduit sections are preferably applied to the first film in a cross-shaped or star-shaped arrangement. In the process, it is possible for the at least three conduit sections arranged on the first film in a cross-shaped or star-shaped fashion to butt against one another in the center of the first film. However, in respect of increasing the drainage capacity of the wound dressing it may be more advantageous if the at least three conduit sections arranged on the first film in a cross-shaped or star-shaped fashion do not butt against one another in the center of the first film. The conduit sections are ideally arranged such that the ends that face the center of the first film have a spacing b of 0.5 to 7 cm from the respectively adjacently arranged ends. In the case of a cross-shaped or star-shaped arrangement of an even number of conduit sections arranged on the first film, the spacing a of opposite ends of the conduit sections should preferably be between 1 and 12 cm.

In this case, the at least three, preferably at least four, conduit sections extend from the central region of the first film to the peripheral region of the first film, more particularly to the edge of the first film. However, in the context of an alternative embodiment, it may be advantageous if the conduit sections do not extend to the edge of the first film such that there is an edge region of the first film which does not have applied conduit sections. This is because the edge region of the wound dressing typically is carefully inserted between the abdominal wall and the wound bed after application on the wound bed. An edge region of the first film as described above, to which no conduit sections have been applied, would then be more particularly provided in this alternative embodiment for insertion between the abdominal wall and the wound bed.

According to a very advantageous embodiment of the invention, the wound dressing comprises a second flexible film, wherein the second film is provided for application on the side of the composite of first film and the at least three conduit sections that faces away from the wound during use. The second film is preferably made of a material that is fluid-impermeable to the greatest possible extent. In order to produce the second flexible film, use can in particular be made of the materials and finished products already mentioned above in the context of the first flexible film, for example thermoplastic films, more particularly films made of EVA, PU, PE, PET, is PTFE, PVC, TPE, silicone or a mixture thereof.

The second film is attached in a non-detachable fashion to the composite of first film and conduit sections, for example by adhesive bonding or welding. The conduit sections and/or those regions of the first flexible film which are localized between the conduit sections come into question as attachment points.

A second flexible film applied to the composite of first film and conduit sections can contribute to improve further the stability and manageability of the wound dressing. As a result of a suitable selection of first film and second film it is possible to match the permeability of the wound dressing for wound fluid, and also the atraumatic properties of the wound dressing, to the respective therapeutic requirements.

According to a first advantageous embodiment, the second film is embodied to be impermeable to fluid to the greatest possible extent and merely has a single opening, the latter preferably being arranged in the central region.

According to a further embodiment of the wound dressing, the second film has a multiplicity of openings distributed across the area thereof, which openings are suitable for passing fluids through them. In the process, it may be advantageous if the openings present in the first film and the openings present in the second film are arranged such that the openings are not congruent to one another to the greatest possible extent. In this context “not congruent to one another to the greatest possible extent” is understood to mean that openings in the first and second film that are congruent to one another at best result by chance and are only present in small numbers. In particular, at least 90%, preferably 95%, of the openings in the first film should not in the process be congruent to an opening in the second film.

The second film can be arranged in different manners on the composite of first film and conduit sections. Exemplary embodiments will be presented below, with further arrangements being feasible and comprised by the invention.

According to a first embodiment, the second film is adhesively bonded to the at least three conduit sections such that a stable composite of first film, conduit sections and second film is created. This can easily be implemented from a technical point of view, for example by virtue of coating the surface of each of the conduit sections with an adhesive and then placing the second flexible film thereon.

According to a further embodiment, the second film is adhesively bonded directly to the first film, but not to the conduit section. Here, the first film can be bonded to the second film over an area, in a punctiform fashion or in a line-shaped fashion. It is also feasible that first film and second film are not adhesively bonded but rather form a non-detachable connection as a result of being welded together. In particular, the welding can be punctiform or along seams. A direct, non-detachable connection between first and second film results in a particularly stable arrangement.

According to a further embodiment, it is also possible to adhesively bond the second film to the first film and to the conduit section. This results in a particularly stable composite.

According to a particularly advantageous embodiment, the second film is attached to the first film by punctiform adhesive bonding or welding of the films, with provision being made for a multiplicity of adhesion points distributed across the area. In the case of such a connection between first and second film, a fluid-conducting interspace is created between the films, which can promote the drainage of wound exudate in an advantageous fashion. In the particularly advantageous embodiment of the invention mentioned here, the conduit sections are situated in a pocket between the two films and are held on the first film in this manner. In this case, the welding of first and second film serves as attachment means for the conduit sections such that it is possible to dispense with adhesively bonding the conduit sections to the first film.

In order to maintain the drainage capacity of the wound dressing during the negative-pressure treatment, it is proposed in the context of a further advantageous embodiment of the invention to provide the film materials comprised by the wound is dressing with a substance with an anticoagulating effect. As a result of this, it is possible where necessary to reduce a blockage of the openings or—if present—of the interspace formed by the first and second film. According to a particularly advantageous embodiment, the film material has a coating of or impregnation with a substance with an anticoagulating effect (for example Heparin or another substance with anticoagulating effect usually used for coating medical surfaces or tubules).

According to a particularly advantageous embodiment of the invention, the wound dressing comprises a connection element, which can be applied to the second side of the first flexible film and to which the end sections of the at least three conduit sections can be attached in a non-detachable fashion. The connection element is placed onto the first film or attached thereto in a non-detachable fashion, preferably in the central region thereof. The connection element expediently comprises a plastic, e.g. of EVA, PU, PVC, PE, PET, PTFE, TPE, silicone or a mixture thereof. Here, in respect of the application it is advantageous if the connection element overall has a soft and flexible design. The connection element should preferably consist of an elastomeric material which has a Shore A hardness of at most 80, preferably at most 70, and more particularly at most 60.

The connection element makes it easier to position correctly the at least three conduit sections in the manner of a supporting plate.

According to a first possible embodiment, the ends or end regions of the conduit sections are attached to the connection element in the manner of a supporting plate such that the conduit sections are present separated from one another. Accordingly, this is brought about by virtue of the ends of the conduit sections not touching one another. Accordingly, the connection element does not form a continuous cavity connecting the present conduit sections in a fluid-conducting manner.

At least three conduit sections are preferably attached to the connection element, with the at least three conduit sections being present in a cross-shaped or star-shaped arrangement.

According to a second feasible embodiment, the connection element forms a central cavity in the manner of a flat box, with the central cavity being connected to the lumina of the conduit sections in a fluid-conducting manner via an open end. The cavity of the connection element has passages on the side that faces away from the wound in order to be able to establish fluid communication with the negative-pressure port. The conduction plate can have a round, square or polygonal design and has a diameter of at least 1 cm and at most 12 cm, more particularly at least 2 cm and at most 10 cm. At least three conduit sections are preferably connected to the cavity of the flat box in a sealing manner, with the at least three conduit sections being present in a cross-shaped or star-shaped arrangement.

If the wound dressing is used to cover large-area wounds in the abdominal region, the edge region of the first film or the edge region of the composite of first film, conduit sections and—if present—second film is usually introduced between the abdominal wall and the wound bed or on the greater omentum. To this end, use is typically made of a flat surgical instrument, for example a stomach or intestinal spatula. Applying the edge region of the wound dressing between abdominal wall and wound bed is a great technical challenge for the surgeon and is accompanied by a risk of injury to the internal organs. In the process, it was, in particular, found to be difficult to apply the edge region of the wound dressing under the abdominal wall without folding the former. It was now found that the wound dressing according to the invention can be introduced into the region between internal organs (or greater omentum) and abdominal wall in a substantially safer, faster and at the same time largely fold-free fashion if the wound dressing has at least one pocket predominantly open to the center of the wound dressing on the side that faces away from the wound during use. To this end, the user can insert a flat surgical instrument, e.g. a stomach and intestinal spatula, into the pocket and thereupon carefully insert the wound dressing, which is temporarily held on the spatula by the pocket, under the abdominal wall. After the edge region of the wound dressing has been inserted under the abdominal wall, the spatula is once again pulled out of the pocket. The wound dressing preferably comprises a multiplicity of pockets. It is then possible—successively or simultaneously—to insert a plurality of peripheral portions of the is wound dressing under the abdominal wall provided that provision is made on the wound dressing for appropriately arranged pockets. Instead of an instrument, the user can also insert one or more fingers into the at least one pocket for applying the wound dressing.

According to the invention, as per the particularly advantageous embodiment presented here, the wound dressing therefore comprises at least one pocket that is open predominantly toward the center of the wound dressing, simplifying the uniform application and laying out of the wound dressing on the wound bed. Here, the at least one pocket is preferably arranged situated toward the edge on the wound dressing. Here, situated toward the edge is understood to mean that the pocket is primarily attached to the peripheral component of the wound dressing. The spacing between the outer edge of the pocket and the edge of the wound dressing could for example be 0 cm to 10 cm on a circular wound dressing with a diameter of 45 cm in the case of an arrangement referred to within this meaning as situated toward the edge. The depth of the at least one pocket is preferably at most 15 cm, more particularly at most 10 cm. The at least one pocket is provided for application on the side that faces away from the wound during use (wound-distant side) of that film ply that is present facing away from the wound during use: to the extent that the wound dressing merely comprises a first flexible film, the pocket is provided for application on the second side of the first film. To the extent that the wound dressing merely consists of two film plies, i.e. of a first flexible film and a second flexible film, the pocket is generally provided for application to the second side of the second film. An exception may occur in this context if the second film only lies over a portion of the first film. In this case it would also be possible to apply the pocket on a portion of the first film not covered by the second film. To the extent that the wound dressing comprises one or more further flexible film plies, the pocket is generally provided for application on the wound-distant side of the film ply oriented at the greatest distance from the wound. This means that the pocket is always provided on the wound-distant side on the wound dressing.

Pockets that are suitable within the scope of the invention can be envisaged in a multiplicity of shapes. What is essential in this case is that there is an opening which is predominantly oriented toward the center of the wound dressing and into is which the surgical instrument, more particularly a stomach and intestinal spatula, can be inserted or hooked. It is furthermore essential that a side of the pocket opposite to the opening is able to provide support to the inserted or hooked-in surgical instrument. The easiest way of implementing such a support is by a seam lying opposite to the opening of the pocket. The opening of the pocket should predominantly be oriented toward the center of the wound dressing such that a surgical instrument moved outward from the center of the wound dressing can be inserted into the opening and can find support therein.

An opening of the pocket predominantly oriented toward the center of the wound dressing can likewise simplify a manual application of the wound dressing if the user wishes to insert a finger instead of a surgical instrument into the at least one pocket.

In respect of its size and shape, the pocket should be matched to the surgical instrument or optionally to a human finger. By way of example, the pocket can substantially have the shape of a rectangle, a trapezium, a semicircle, a triangle, an annulus or an annular sector, with further shapes being comprised which are not described here in any more detail but emerge for a person skilled in the art from the holding function of the pocket.

The depth of the pocket, i.e. the spacing of the opening of the pocket from the opposite edge of the pocket, should preferably be at most 15 cm, more particularly at most 10 cm. The depth should be at least 0.3 cm, but preferably at least 1 cm, so that the surgical instrument inserted into the pocket or a finger optionally inserted into the pocket cannot slip out of the pocket during the application of the wound dressing. A pocket which is suitable according to the invention therefore preferably has a depth of 1 cm to 10 cm.

In respect of the width b of the pocket, it is likewise necessary to match the former to the surgical instrument that is used when applying the wound dressing, which is why the width b of the pocket must at least equal the width of the instrument is because otherwise the latter cannot be inserted into the pocket. However, it is advantageous to design the pocket to be substantially wider than the instrument because it is easier to insert the instrument into the pocket in the case of a pocket designed like this. In this case, the user does not need to take care to find a comparatively narrow opening of the pocket, which may be difficult in the case of a strongly bleeding wound. In general, the width b of the pocket is at least 1 cm, preferably at least 2 cm.

According to the invention, the wound dressing comprises at least one pocket present on the side of the wound dressing that faces away from the wound during use (wound-distant side). However, it proves to be particularly advantageous for the application of the wound dressing if the wound dressing comprises a multiplicity of pockets, which are applied at various points on the wound dressing. In this case, it is proposed that the wound dressing comprises at least 3 pockets, preferably at least 4, more particularly at least 6 pockets. The pockets should be applied to the wound dressing with a spacing between one another that is uniform to the greatest possible extent. In particular, the pockets can be present arranged on one or more concentric circles which encircle the center point of the wound dressing, with the pockets preferably being present on each circle distributed with a uniform spacing between one another. The pockets are preferably present on at least 30% of the circumference of the aforementioned concentric circle, more particularly on at least 50% of the circumference.

In respect of the possibility of the user cutting the wound dressing to size, it is particularly advantageous here if the pockets are present on the wound dressing arranged on a plurality of concentric circles which encircle the center point of the wound dressing. If the pockets arranged further on the outside are cut off or destroyed during the cutting to size in the case of such an arrangement, it is still possible to use the pockets present further on the inside when applying the wound dressing.

A preferred embodiment of the wound dressing according to the invention comprises at least one pocket, wherein the pocket is formed by the application of is areal sections of material, more particularly pieces of film, on the side of the wound dressing that faces away from the wound during use. In particular, the pocket can be attached to the wound dressing by adhesive bonding, thermal welding, pressing or ultrasound welding. The wound dressing is attached at the edge of the material section such that an outer seam is produced. Here, the edge of the material section predominantly oriented toward the center of the wound dressing is not connected to the wound dressing such that there is an opening available for inserting a surgical instrument or a finger. In terms of its design, such a pocket is similar to a shirt pocket, i.e. a pocket sealed on three sides by a seam, which has an opening into which an object can be inserted. Here, the inner surface of the pocket that points toward the wound during use is formed by the film portion of the wound dressing arranged on the wound-distant side, while the inner surface of the pocket that points away from the wound during use is formed by the applied material section. Such a pocket can be produced in a simple and cost-effective manner.

According to an alternative and likewise very advantageous embodiment of the invention, the wound dressing has one or more pockets on the wound-distant side, which pockets are made of a material section in the form of an annulus. A pocket embodied thus is particularly suitable for application to a circular abdominal wound dressing. On its outer circumference, the material section with the shape of an annulus is attached to the wound dressing such that a circular seam is formed. Each material section respectively forms a single pocket, which encircles the center of the wound dressing and has an opening pointing toward the center of the wound dressing. Here, the outer circumference of the annulus may at most have the dimensions of the wound dressing. The inner circumference of the annulus should preferably be selected such that a pocket is formed with a depth of at most 15 cm, more particularly of at most 10 cm. It is possible to apply a plurality of such pockets, arranged concentrically, onto the wound dressing. This simplifies the application of a wound dressing which is fitted to the wound dimension by cutting to size: after destroying a pocket applied further to the outside by cutting to size, a pocket available further to the inside can be used during the application of the wound dressing.

Here the aforementioned embodiment is not restricted to wound dressings with a precise circular design, but rather can likewise be embodied in the case of oval abdominal wound dressings if the material section is suitably adapted.

A further preferred embodiment of the wound dressing according to the invention comprises at least one pocket which is embodied like a sack or a cone and provided for application to the side of the wound dressing that faces away from the wound during use. Here, the pocket should be applied to the wound dressing such that the opening of the former is present oriented predominantly to the center of the wound dressing. The pocket is preferably made of a film material and should furthermore preferably have a depth a of at most 15 cm, more particularly of at most 10 cm. In particular, the pocket can be attached to the wound dressing by adhesive bonding, thermal welding, pressing or ultrasound welding. The inner surface of the pocket that points toward the wound during use is formed by a first material section of the cone in this embodiment, while the inner surface of the pocket that points away from the wound during use is formed by a second material section of the cone.

According to a further preferred embodiment, the wound dressing according to the invention comprises at least one pocket, with the pocket being formed by folding back a film portion of the wound dressing. Here, the pocket is more particularly formed by folding back the first film and/or the second flexible film.

In order to ensure a sufficient drainage capacity of the wound dressing, provision can optionally be made for the pocket to be made of a material, more particularly a film material, which has a multiplicity of openings distributed across the area. The openings present in the material section provided for forming a pocket should preferably have at least 0.1% and at most 25% of the areal extent, preferably at least 10% and at most 22% of the areal extent of the material section.

In order to apply a wound dressing according to the invention, which comprises at least one of the aforementioned pockets, the user can insert a flat surgical instrument, e.g. a stomach and intestinal spatula, into the at least one pocket and thereupon carefully insert the wound dressing, which is temporarily held on the is spatula by the pocket, under the abdominal wall. Alternatively, it would also be possible to insert one or more fingers into the at least one pocket, instead of an instrument, for applying the wound dressing.

In practice, it furthermore is found to be very advantageous if, on the side that points away from the wound, the wound dressing comprises one or more liquid-permeable layers for application onto the side of the wound dressing that faces away from the wound during use. According to a further embodiment of the invention, the wound dressing therefore comprises one or more liquid-permeable layers for application onto the side of the composite of first film and conduit sections, or of the composite of first film, conduit sections and second film, that faces away from the wound during use. The liquid-permeable layer preferably comprises a porous foam, more particularly a porous polymer foam. An open-cell polymer foam is particularly suitable in this case. Within the scope of this application, the term open-cell means that, compared to the total number of cells, there are at least 60% open cells, preferably at least 90% open cells, more preferably at least 98% open cells, more particularly substantially 100% open cells in the foam (c).

By way of example, suitable materials for a porous foam comprise polyurethane, polyurethane-polyurea copolymers, polyvinyl alcohol (PVA) or silicone.

As an alternative, or in addition thereto, the liquid-permeable layer can comprise textile materials such as wovens or non-wovens, for example a non-woven material of synthetic polymers such as polyamide, polyester or polypropylene.

In the context of the present invention, the porous foams described in the German patent application DE102010034819.8 (not yet published at the time of the present application) can be used in a particularly advantageous fashion to produce the one or more liquid-permeable layers. Reference is herewith made to the content of the German patent application DE102010034819.8. The foams described in DE102010034819.8 do not release foam particles, or only release the latter to a small extent, during the possibly required cutting to size for matching the wound shape. Released foam particles that reach the wound can irritate the wound and have an adverse effect on wound healing.

In particularly advantageous fashion, use can be made of the open-cell polyurethane foam VivanoMed® Foam, distributed by the applicant Paul Hartmann AG (Heidenheim, Germany), as a liquid-permeable layer.

The at least one further liquid-permeable layer can improve the softness and tolerance of the wound dressing and can offer an additional contribution to draining wound exudate.

Furthermore, for the purpose of promoting the primary wound closure, it is more particularly very advantageous to apply a porous polymer foam such that the foam is in direct contact with the wound edges.

The at least one further liquid-permeable layer has a thickness of between 2 mm and 50 mm, preferably of between 3 mm and 30 mm.

The further liquid-permeable layer, which preferably comprises a porous polymer foam, can in this case cover the whole area of the composite of conduit sections and first film (or, if present, of the composite of first film, conduit sections and second film). The liquid-permeable layer is preferably only present on a central portion of the area of the first or—if present—second film. Particularly when using the wound dressing as temporary wound closure, it was found to be expedient if a porous polymer foam is applied such that the edges of the foam are in direct contact with the wound edges.

If the conduit sections are connected by a centrally arranged connection element, it can be advantageous for the further liquid-permeable layer to have a corresponding cut-out for holding the connection element. Here, the cut-out can be embodied merely as a recess or alternatively as a continuous opening.

In the context of the present invention, it is possible to provide more than one liquid-permeable layer, more particularly more than one layer of a porous polymer foam, on the side of the composite of first film and conduit sections or of the composite of first film, conduit sections and second film that faces away from the wound during use. Here, the plurality of layers can have different dimensions and be present in different thicknesses. For negative-pressure therapy of a typical abdominal wound it was found to be advantageous if two layers of an open-cell polyurethane foam with a layer thickness of respectively 16 mm are applied.

The wound dressing according to the invention can furthermore comprise an air-impermeable cover material for airtight closure of the wound and the surroundings of the wound, with the cover material preferably having an adhesive edge for attaching the cover material to the intact skin surrounding the wound. Here, “airtight closure” should not be understood to mean that there is no gas exchange between the wound space and the surroundings thereof. Rather, “airtight closure” in this context means that, taking into account the utilized negative-pressure source, the negative pressure required for the negative-pressure wound therapy can be maintained. It is therefore also possible for use to be made of cover materials which have a small gas permeability, as long as the negative pressure required for the negative-pressure therapy can be maintained.

The cover material is attached in the surroundings of the wound or on the wound edge such that an airtight wound closure is ensured. In the process, it can be expedient if the cover material is equipped to be self-adhesive over the whole area or has a self-adhesive edge. Alternatively, attaching and sealing can for example be brought about using an adhesive film, a liquid adhesive or a sealing compound.

In a preferred embodiment of the invention, the cover material for airtight closure of the wound comprises a water-insoluble polymer or a metal film.

In a particularly preferred embodiment of the invention, the water-insoluble polymer is polyurethane, polyester, polypropylene, polyethylene, polyamide or is polyvinyl chloride, polyorganosiloxane (silicone) or a mixture thereof.

It is also possible to use finished products which have the aforementioned properties as cover material.

A polyurethane film branded Hydrofilm® (Paul Hartmann AG, Germany) or Visulin® (Paul Hartmann AG, Germany) was found to be a particularly suitable cover material for the device according to the invention.

In the context of the wound dressing according to the invention, a negative-pressure port is furthermore claimed for the functional connection of the wound space to a negative-pressure source situated outside of the wound dressing, with the negative-pressure port being designed such that negative pressure can be set in the wound space and liquids can be suctioned out of the wound space. When using the wound dressing in the negative-pressure wound therapy, the negative-pressure port is preferably applied to the side of the air-impermeable cover material that faces away from the wound, with the cover material having suitable openings. A person skilled in the art also knows of negative-pressure ports by the name “port”. The negative-pressure port usually comprises a connection line and a negative-pressure adapter in order to be connectible to the further components of the negative-pressure system.

According to a further preferred embodiment, cover material and the means for the functional connection of the wound space to a negative-pressure source situated outside of the cover material are already provided in a ready-made interconnected fashion. It is very particularly preferred for this embodiment to contain a film made of one or more water-insoluble polymers, which has a self-adhesive edge, because this arrangement makes it substantially easier to apply the bandage.

The negative-pressure ports disclosed in the patent applications WO2011091947, WO2011091952 and WO2011076340, as well as in the German patent application DE102011108726.9 (not published at the time of the present application), are particularly suitable for the wound dressing described in the present invention.

According to an alternative embodiment, the functional connection of the wound space to a negative-pressure source situated outside of the cover material can be established using at least one connection line. The at least one connection line can be routed through the cover material or routed under the edge of the cover material. The passage point is to be sealed in an airtight manner in both cases so that the desired negative pressure can be maintained in the bandage. By way of example, an adhesive film, an adhesive compound or an adhesive strip are suitable as a sealing means.

By way of example, the connection line can be a tube, a pipe or another body with a cavity. By way of example, a silicone drainage tube is a suitable tube.

The wound dressing according to the invention can furthermore comprise a means such that the negative pressure actually present within the device can be monitored and set, if required. The means can be in the wound space or at another suitable place.

Alternatively, it is also possible to attach a pressure sensor in the negative-pressure line between wound bandage and the negative-pressure source.

It is envisaged that the aforementioned components are provided to the medical practitioners and specialist staff treating the wound in the form of a ready-made set (“kit”). The invention therefore also relates to a ready-made set for negative-pressure wound treatment, comprising

a) a wound dressing according to one or more of claims 1 to 17,

b) at least one liquid-permeable layer for application on the side of the wound dressing that faces away from the wound during use, wherein the at least one liquid-permeable layer preferably comprises one or more areal cushions of a porous polymer foam, more particularly of PU, PVA or silicone,

c) an air-impermeable cover material for airtight closure of the wound and the surroundings of the wound, wherein the cover material preferably has an adhesive is edge,

d) a negative-pressure connection means for the functional connection of the wound space to a negative-pressure source situated outside of the cover material such that negative pressure can be set in the wound space and liquids can be suctioned out of the wound space, wherein the negative-pressure connection means is preferably provided for application to the outer side of the cover material that faces away from the wound during use,

and wherein components a) to d) can be present in sterile packaged form.

Negative-pressure ports particularly suitable for the ready-made set are described in the aforementioned patent applications with application numbers WO2011091947, WO2011091952, WO2011076340 and DE102011108726.9 (not published at the time of the present application).

The set can furthermore contain optional components such as e.g. one or more additional areal elements of a liquid-permeable layer, adhesive means for fixing the bandage, sealing means for establishing an air-impermeable seal of the bandage, pressure sensors, connection elements for pressure sensors, tubes, connection pieces for tubes, disinfection means, skin-care products, pharmaceutical preparations or instructions for use.

The set furthermore preferably comprises a ready-made negative-pressure unit. The negative-pressure unit can contain components such as e.g. a pump, one or more liquid containers, a control unit, a power supply, electric connection means, and tubes. The negative-pressure unit can also contain a device for the functional connection of the negative-pressure bandage to an available stationary negative-pressure source.

Negative-pressure units particularly suitable for the ready-made set are described in the patent applications WO2011018133 and WO2011018132. A negative-pressure unit particularly suitable for the set is commercially available under the name VivanoTec® (producer Paul Hartmann AG, Heidenheim, Germany).

All components for which it is necessary from a medical point of view are preferably made available in sterile packaged form. The advantage of the ready-made set consists of the fact that the negative-pressure bandage can be applied in a quick, standardized and uncomplicated fashion. A further advantage consists of the fact that all components of the set used in the region of the wound can be provided in an already sterilized fashion.

### KEY TO THE FIGURES

1. First flexible film with a multiplicity of openings distributed across the area
2. Conduit section
3. Cavity (lumen) in a conduit section
4. Second flexible film
5. Opening situated at the end of a conduit section
6. Opening in the first flexible film
7. Adhesion or attachment point between first and second film
8. Connection element
10. Wound dressing for use in negative-pressure wound therapy, in particular for treatment of abdominal wounds
11. Liquid-permeable layer
12. Interspace between wound bed (e.g. exposed internal organs or the greater omentum) and abdominal wall
13. Wound bed, more particularly exposed internal organs or the greater omentum
14. Abdominal wall
15. Wound edge
16. Air-impermeable cover film
17. Opening in the cover film
18. Negative-pressure connection means (port)
19. Negative-pressure line
20. Can for wound exudate
21. Negative-pressure source
22. Adhesive layer which connects the first film to a conduit section
25. Pocket open toward the center of the wound dressing
26. Seam or adhesive region between pocket and first or second flexible film
27. Opening in the pocket
28. Lumen of the negative-pressure line
40. Device for use in negative-pressure wound therapy, in particular for treatment of abdominal wounds
50. Measuring device for testing the drainage capacity of a wound dressing
51. First hollow body
52. Second hollow body
53. First opening in the second hollow body
54. Second opening in the second hollow body
521. Additional ply of a flexible material
531. Fluid source
532. Conduit for supplying blood substitute solution
533. Negative-pressure source
534. Collection can
535. Drainage line
536. Negative-pressure port (port)
537. First flexible film
538. Foam ply
539. Self-adhesive cover film
540. Measuring device for fluid volume

### BRIEF DESCRIPTION OF THE DRAWINGS

In the following text, the wound dressing according to the invention and the device for negative-pressure wound therapy will be explained in more detail on the basis of schematic drawings (not true-to-scale). However, the invention should not be understood as being restricted to the embodiments illustrated in the drawings or in the description of the drawing. Rather, the device according to the invention also comprises combinations of the individual features of the alternative forms.

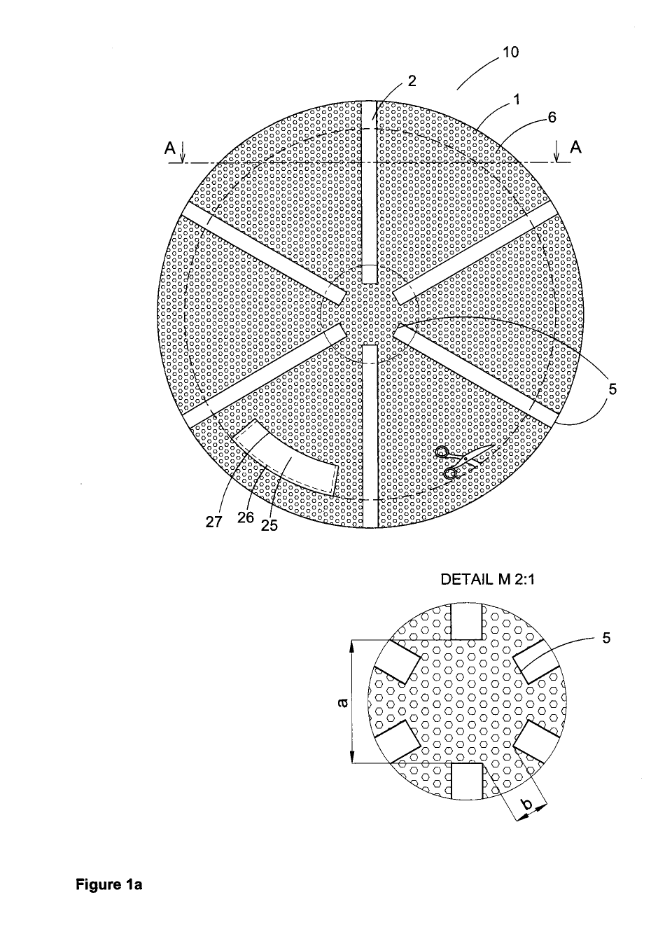

FIG. 1a shows an embodiment of the wound dressing according to the invention for use in negative-pressure therapy, in a plan view of the side of the wound dressing that faces away from the wound.

FIG. 1b shows a further embodiment of the wound dressing according to the is invention for use in negative-pressure therapy, in a plan view of the side of the wound dressing that faces away from the wound.

FIG. 1c shows a further embodiment of the wound dressing according to the invention for use in negative-pressure therapy, in a plan view of the side of the wound dressing that faces away from the wound.

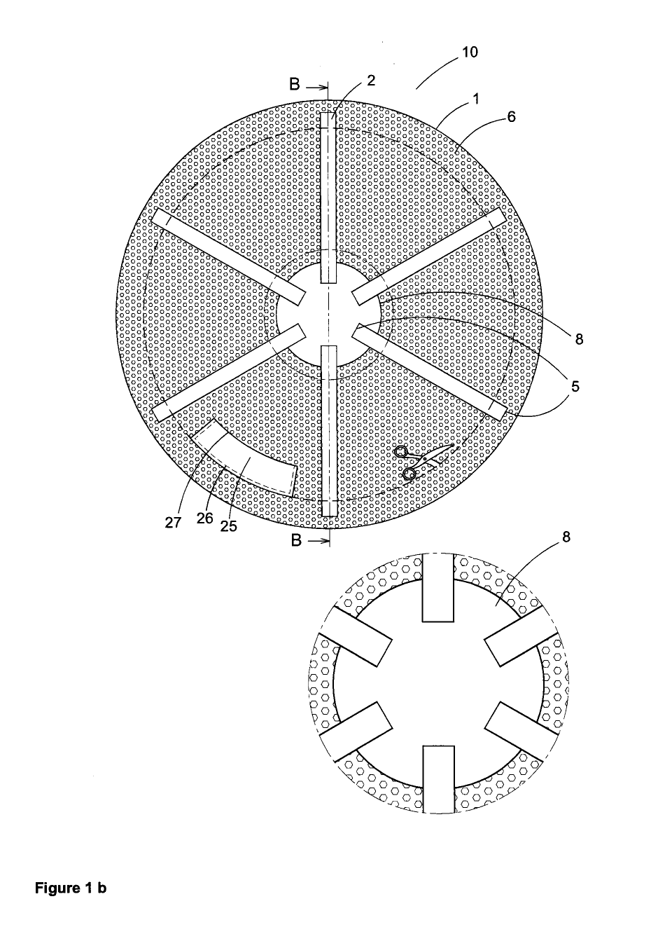

FIG. 2 shows a cross section along the line A-A through the wound dressing illustrated in FIG. 1a.

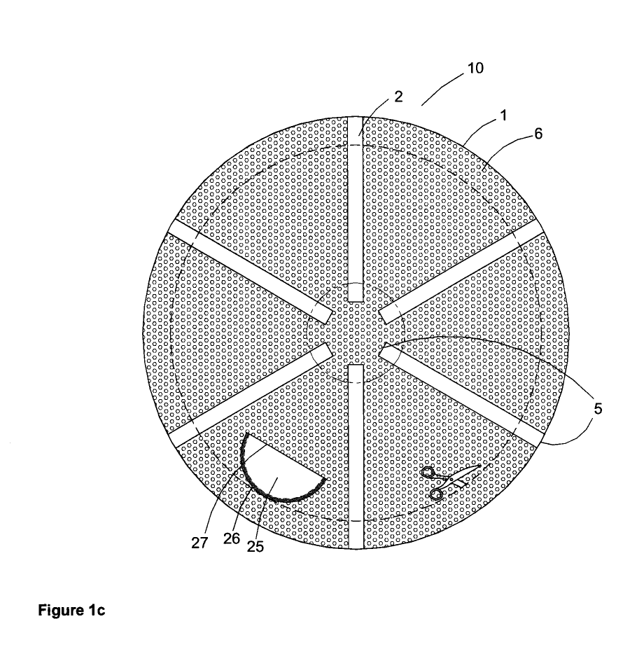

FIG. 3 shows a cross section of a further embodiment of the wound dressing according to the invention. The wound dressing comprises a first flexible film and additionally a second flexible film. The cut was made through a peripheral region of the wound dressing, corresponding to the line A-A in FIG. 1a.

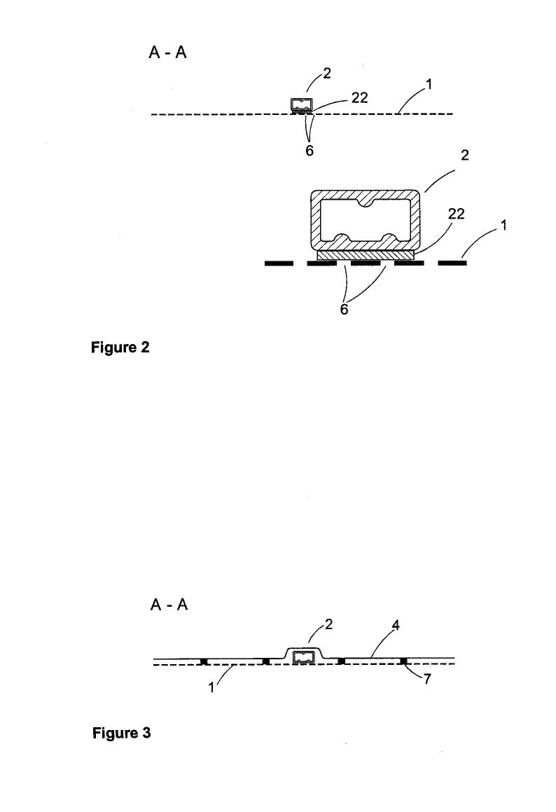

FIG. 4 shows, in a cross section, a preferred embodiment of a device applied to an abdominal wound, for use in negative-pressure wound therapy. The cut was made through the center of the wound dressing, corresponding to the line B-B in FIG. 1 b.

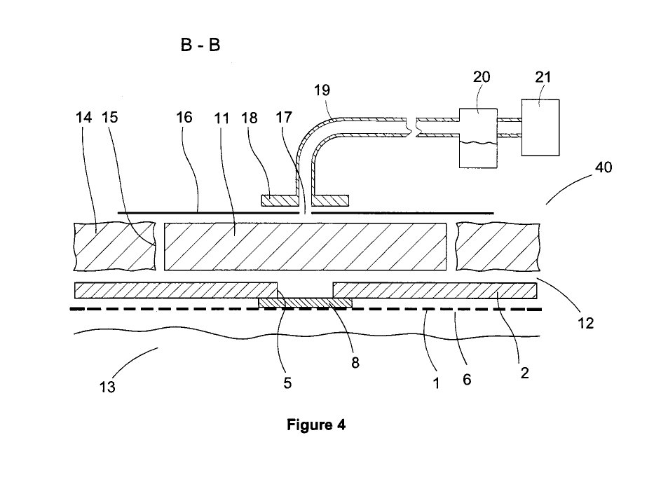

FIG. 5 shows a measuring device for wound bandages (“wound simulator”), including a cross section of an NPWT abdominal bandage to be tested.

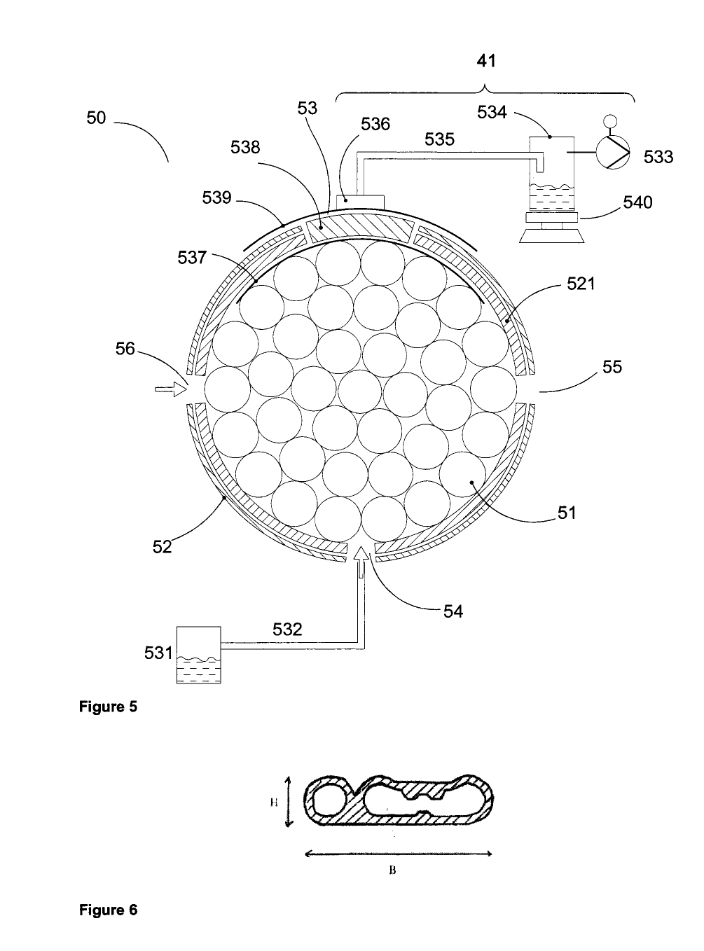

FIG. 6 shows a cross section of an embodiment of a conduit section.

### DETAILED DESCRIPTION OF THE INVENTION

The embodiments of the wound dressing illustrated in FIG. 1 a and FIG. 1 b respectively comprise a first flexible film (1), onto which conduit sections (2) have been applied. The first flexible film (1) has a first side and a second side, wherein the first side is provided for application on the wound bed (see FIG. 4, reference sign 13), in particular on exposed internal organs or on the greater omentum, and hence can serve as wound contact layer. The first film (1) furthermore has a multiplicity of openings (6) distributed across the area. In the embodiment of the invention illustrated in FIG. 1 a and FIG. 1 b, provision is made for circular openings (holes), wherein the sum of the open area of the openings (6) present in the first film (1) is approximately 14% of the areal extent of the film. However, the sum of the open area of the openings (6) present in the film (1) should be at least 0.1% and at most 25% of the areal extent, preferably at least 10% and at most 18% of the areal extent, more particularly at least 13% and at most 15% of the areal extent of the film. By way of example, such openings (6) can be produced by stamping. In the embodiments schematically illustrated in FIG. 1 a and FIG. 1 b, respectively six conduit sections (2) made of a flexible elastomeric material with a thickness (H) of approximately 6 mm are arranged on the first flexible film (1) in a star-shaped fashion on the first flexible film (1). The width (B) of the conduit sections (2) is approximately 20 mm. With the exception of the openings (5) situated at the ends, the conduit sections (2) have no further openings. The conduit sections (2) are connected to the first film (1) in a non-detachable fashion, for example by adhesive bonding. Here, the conduit section (2) has a flat design and, with at least 10% of its area projected is perpendicularly onto the first film (1), is connected to said film in a non-detachable and areal fashion. The conduit section (2) is made of an elastomeric material which has a Shore A hardness of 60. Suitable materials comprise EVA, PU, PVC, PE, PET, PTFE, TPE, silicone or a mixture thereof. In any case, the Shore A hardness should be at least 20 and at most 70, preferably at least 30 and at most 65; in particular, a conduit section (2) should have a Shore A hardness of at least 40 and at most 62. A conduit section (2) can be obtained by cutting to length a longer drainage tube which is provided for medical purposes.

As indicated by the scissors symbol in FIG. 1 a and FIG. 1 b, the wound dressing can be matched to the size required for the treatment of the wound by cutting to size, wherein both the first film (1) and optionally one or more conduit sections (2) can be severed.

In the embodiment of the invention illustrated schematically in FIG. 1 a, the end pieces, having an opening (5) situated at the ends, of the conduit sections (2) arranged on the first film (1) in a star-shaped fashion are spaced apart from one another. The spacing, characterized by the reference sign b in FIG. 1 a, between adjacent conduit sections (2) respectively is approximately 2 cm in the embodiment shown in an exemplary fashion; however, it can also be less than or greater than this. A spacing b of 0.5 to 7 cm proved to be expedient. The spacing a of the ends of the conduit sections (2), arranged opposite one another on the first film (1), pointing toward the center of the wound dressing emerges from the number of the conduit sections present and the arrangement thereof. In the embodiment presented here in an exemplary fashion it is approximately 8 cm.

However, it is also possible for the conduit sections (2) arranged on the first film (1) in a cross-shaped or star-shaped fashion to butt against one another in the center of the first film (1) (not illustrated).

The ends of the six conduit sections (2), which are oriented toward the outside and hence toward the periphery of the wound dressing and which likewise have an opening (5) situated at the end, extend up to the edge of the first film (1) in the embodiment illustrated in FIG. 1 a. However, the ends (5) of the conduit sections (2) oriented to the outside can also be recessed compared to the edge of the first film (1), as shown in FIG. 1 b. By way of example, in this case there may be a spacing of 1 to 10 cm present between the end of a conduit section (2) and the edge of the first film (1).

FIG. 1 a illustrates a pocket (25), which can simplify uniform application and laying out of the bandage on exposed internal organs or on the greater omentum. Such a pocket (25) can be formed by the application of additional pieces of material, more particularly pieces of film, onto the first film (1). A pocket (25) could also be formed by folding in the first film (1). On its edges, the pocket (25) is attached to the first film by a seam or an adhesive region (26), with, however, the side of the pocket pointing toward the center of the wound dressing having an opening (27). The treating medical practitioner can insert a stomach or intestinal spatula or optionally a finger into the opening pointing toward the center of the wound dressing and can thereupon carefully introduce the edge region of the wound dressing between abdominal wall and the wound bed. The pocket (25) shown in FIG. 1 a has the shape of an annular sector, with alternative embodiments of the pocket (25), for example the form illustrated in FIG. 1 c, with an opening likewise pointing toward the center of the wound dressing being feasible. In FIG. 1 a, one pocket (25) only is illustrated in an exemplary fashion. In practice, a multiplicity of such pockets (25) are provided, preferably on at least 30% of the circumference of the wound dressing. In the example shown in FIG. 1 a, it would be advantageous for a pocket (25) to be attached in each segment formed between the six conduit sections (2) such that a total of six pockets (25) are present (not illustrated). It would also be possible to attach one or more additional rows of pockets (25) arranged in concentric circles (not illustrated in FIG. 1 a) such that there still are pockets arranged further inward, even after cutting the wound dressing to size (to the extent that pockets are partly or wholly cut off in the process). The depth of a pocket (25) provided for simplifying the application of the wound dressing expediently is at least 0.5 cm and at most 15 cm.

In addition to the components present in FIG. 1 a, the embodiment shown in FIG. 1 b comprises a plate-shaped connection element (8) with a diameter of 5 cm, which can be applied to the second side of the first flexible film (1). The end regions, is having an opening (5) situated at the ends, of the conduit sections (2) can be attached in a non-detachable fashion to the connection element (8) which is preferably produced from an elastomeric material. The conduit sections (2) are preferably attached to the side of the connection element (8) that points away from the wound during use. The end pieces, having an opening (5) situated at the ends, of the conduit sections (2) arranged on the first film (1) in a star-shaped fashion can butt against one another in the center or, as shown in FIG. 1 b, can be spaced apart from one another.

FIG. 1 c shows a further embodiment of the wound dressing according to the invention, illustrating an alternative and likewise advantageous embodiment of the pocket (25) but otherwise having the structural elements illustrated in FIG. 1 a. The pocket (25) of the embodiment shown in FIG. 1 c has an arc-shaped seam. The opening (27) pointing to the center of the wound dressing is provided for introducing a stomach or intestinal spatula or optionally a finger. Like in FIG. 1 a, only one pocket (25) is illustrated in an exemplary fashion. However, in practice, a multiplicity of such pockets (25) should be present, preferably in each of the six segments formed between the conduit sections (2) (not illustrated).

FIG. 2 shows a cross section (not to scale) along the line A-A through the wound dressing illustrated in FIG. 1 a. A conduit section (2) with a substantially rectangular cross section is attached in a non-detachable fashion on a first flexible film (1) which has a multiplicity of openings (6) arranged distributed across the area. In the case illustrated in exemplary fashion, the conduit section (2) only has a single continuous cavity (3). On the inner side, the conduit section (2) can have bulges or thickenings which serve to stabilize the conduit (2) in negative pressure.

An adhesive layer (22) is situated between the second side of the film (1) and the conduit section (2), by means of which the conduit section (2) is connected in non-detachable and areal fashion to the second side of the first film with approximately 90% of its area projected perpendicularly onto the first film. However, within the scope of the invention alternative means for attaching the conduit section (2) to the film (1) are also possible.

FIG. 3 shows a further embodiment of the wound dressing according to the invention. The wound dressing differs from the embodiment shown in FIG. 2 in that a second flexible film (4) is additionally present, wherein the second film (4) is applied in a non-detachable fashion to the side of the composite of first film (1) and conduit sections (2) that faces away from the wound during use. First flexible film (1) and second flexible film (4) are interconnected in a non-detachable fashion by means of adhesion or attachment points (7). The conduit section (2) is held in a pocket formed by the first film (1) and the second film (4) such that an adhesive layer between conduit section (2) and first film (1) can be dispensed with in the embodiment shown in FIG. 3. The second flexible film (4) thus brings about the attachment of the conduit section (2) on the first film (1). Punctiform adhesive bonding between first film (1) and second film (4) is found to be particularly advantageous because a labyrinth-like cavity is formed between first film (1) and second film (2) and it can further improve the drainage capacity of the wound dressing.

FIG. 4 shows, in a cross section, a device (40), applied to an abdominal wound, for negative-pressure wound therapy. The device comprises a wound dressing (10) according to the invention, as described in more detail above, with a first film (1) and conduit sections (2). The first side of the first film (1) is applied to the wound bed (13), in particular to exposed internal organs or to the greater omentum. First film (1) and conduit sections (2) applied thereon are usually inserted into the interspace formed between wound bed (13) and abdominal wall (14). According to an embodiment of the wound dressing not illustrated in FIG. 4, the first film (1) can have an edge region on which no conduit sections (2) are applied. In this case it is possible that only the first film (1), but no conduit sections (2), are inserted into the interspace formed between wound bed (13) and abdominal wall (14).

The insertion of the wound dressing into the interspace (12) formed between wound bed (13) and abdominal wall (14) can be simplified if provision is made on the wound dressing for pockets (25), which are situated on the edge and open toward the center of the wound dressing (not illustrated in FIG. 4, see FIGS. 1 a-c). The treating medical practitioner can insert a stomach or intestinal spatula or optionally a is finger into the pockets and thereupon insert the edge region of the wound dressing between abdominal wall (14) and the wound bed (13).

On the side of the composite of first film (1) and conduit sections (2) that faces away from the wound during use there is a liquid-permeable layer (11), which more particularly is a porous polymer foam. Use is preferably made of an open-cell polyurethane foam. Depending on the depth of the wound, it is possible for a plurality of layers of the liquid-permeable layer (11) to be present (not illustrated). By cutting to size, the liquid-permeable layer (11) was matched to the size of the wound such that the edge of the liquid-permeable layer (11) is in direct contact with the wound edge (15). It is known that contacting the wound edge (15) with a porous polymer foam (11) promotes the growth of the wound-edge tissue. The device (40) furthermore comprises an air-impermeable cover material (16) for airtight closure of the wound, and also a means (18), more particularly a negative-pressure port (port), that can be applied onto the air-impermeable cover material (16) on the side that faces away from the wound, for the functional connection of the wound space to a negative-pressure source (21) situated outside of the cover material (16). The negative-pressure port (18) is attached in the region of an opening (17) introduced into the air-impermeable cover material (16). When the negative-pressure source (21), which is connected in fluid-conducting fashion to the port (18) via a collection can for wound exudate (20) and a negative-pressure line (19), is in operation, negative pressure can be set in the wound space and liquids can be suctioned out of the wound space. Wound exudate suctioned out of the wound space via the negative-pressure line (19) is collected in the can (20).

When negative pressure is applied, it is possible for wound exudate from the wound bed (13) to reach the liquid-permeable layer (11) via openings (6) in the first film (1). Additionally, wound exudate, particularly from the peripheral region of the wound bed (13), can be guided to the central region of the wound dressing through a conduit section (2) and can there pass into the liquid-permeable layer (11). From the liquid-permeable layer (11), the fluid is transported away through an opening (17) in the cover film (16) to the negative-pressure port (18) and on to the can (20) through the negative-pressure line (19).

Portable negative-pressure units which are particularly suitable in the context of the present invention and separably comprise a can (20) and negative-pressure source (21) in a single unit are described in the aforementioned patent applications with the application numbers WO2011018133 and WO 2011018132. In respect of suitable negative-pressure connection means (18), reference is made to the aforementioned patent applications WO2011091947, WO2011091952, WO2011076340 and DE102011108726.9 (not published at the time of the present application).

FIG. 6 shows, in an exemplary fashion and in a cross section, an embodiment of a conduit section (2) with a thickness (H) and width (B). The conduit section illustrated in FIG. 6 has two continuous cavities which are not interconnected. The ratio of the width B of the conduit section to the thickness H thereof should preferably be at least 1.25. In the example illustrated in FIG. 6, the ratio of the width B of the conduit section to the thickness H thereof is approximately 3.7. In the context of the invention, use can be made of conduit sections with only one continuous cavity or else with a plurality of continuous cavities.

### Production of the Wound Dressing

The invention furthermore relates to a method for producing a wound dressing for negative-pressure therapy, wherein the wound dressing is provided in particular for the treatment of abdominal wounds, said method comprising the following steps:

a) provision of a first flexible film (1) with a first and a second side, with the first side being provided for application on the wound bed (13), more particularly on exposed internal organs or on the greater omentum, and with the film (1) having a multiplicity of openings (6) distributed across the area,

b) provision of at least three conduit sections (2) made of a flexible elastomeric material with a thickness (H) of at most 20 mm, with each of the conduit sections (2) having at least one continuous cavity (3), and with each of the conduit sections (2) having a flat design, and wherein furthermore, with the exception of the two openings (5) situated at the ends, each of the conduit sections (2) has no further openings,

c) optional provision of a connection element (8), to which the end sections of the at least three conduit sections (2) can be attached in a non-detachable fashion and which can be applied to the second side of the first flexible film (1), preferably in a central fashion,

d) optional connection of the end sections of the at least three conduit sections (2) to the connection element (8),

e) application of the at least three conduit sections (2) or the composite of the conduit sections and the connection element (8) to the second side of the first film (1), wherein a cross-shaped or star-shaped arrangement of the conduit sections (2) on the first film (1) is preferred,

f) optional application of a second flexible film (4) onto the composite of first film (1) and conduit sections (2), wherein the second film is applied to the side of the composite that points away from the wound during use,

g) optional application of at least one pocket (25) which is situated on the edge and open predominantly to the center of the wound dressing, wherein the pocket (25) is formed preferably by the application of further pieces of material, more particularly pieces of film, to the second side of the first film (1) or—if present—on the side of the second flexible film (4) that faces away from the wound during use.

In the first film (1) used in step (a), the sum of the open area of the openings (6) present in the film (1) should, where possible, be at least 0.1% and at most 25% of the areal extent, preferably at least 10% and at most 22% of the areal extent of the film.

The diameter of the openings (6) present in the first film (1) should be at least 0.2 mm and at most 0.4 mm, in particular 0.3 mm, with there being at least 150 and at most 300 openings per cm². Advantageously, a film with a mass per unit area of at least 30 g/m² and at most 150 g/m², preferably at least 45 g/m² and at most 95 g/m² and more particularly at least 55 g/m² and at most 65 g/m² can be used as first film (1).

The openings (6) present in the first film (1) can be produced by perforating is the film such that openings are created which are conical or cylindrical to the greatest possible extent and, as a result of this, the film has a smooth side and a roughened side which lies opposite to the smooth side. Alternatively, openings with a three-dimensional structure can already be produced during the production of the film.

In step (e), the conduit sections are then applied preferably to the roughened side such that the smooth side of the first film (1) serves as wound contact layer when the wound dressing is used.

The at least three conduit sections (2) used in step (b) should be made of a material which has a Shore A hardness of at least 20 and of at most 70, preferably a Shore A hardness of at least 30 and of at most 65, more particularly a Shore A hardness of at least 40 and of at most 62. Suitable materials comprise EVA, PU, PVC, PE, PET, PTFE, TPE, silicone or a mixture thereof.

The end sections of the at least three conduit sections (2) can, optionally before the conduit sections (2) are applied to the first film (1), be attached to a connection element (8), for example to a plastic plate, in a cross-shaped or star-shaped arrangement and in a non-detachable fashion (step d). The composite created hereby of conduit sections and connection element (8) can simplify the intended alignment of the conduit sections during the application of the conduit sections (2) onto the first film (1) in step (e). The conduit sections can be attached to the connection element by e.g. adhesive bonding or welding.

The at least three conduit sections (2) or—if present—the composite of conduit sections and connection element (8) are/is applied to the second side of the first film (1) and attached to the first film in a preferably non-detachable fashion. By way of example, this can be brought about by adhesive bonding or welding. According to an alternative embodiment, the conduit sections (2) or—if present—the composite of conduit sections and connection element (8) can also be held on the first film by a second flexible film (4). Here, the second flexible film (4) is connected to the second side of the first film such that the conduit sections or—if present—the composite of conduit sections and connection element (8) are/is attached in a pocket-like manner between the two films. In this case it is advantageous if first and is second film are interconnected in a non-detachable fashion by a multiplicity of punctiform adhesions or weldings.

### Testing the Drainage Capacity of the Wound Dressing

It is possible to test a wound dressing according to the invention, in particular measure the drainage capacity of the wound dressing, using a measuring device (50) for wound bandages. Such a measuring device is described in the German patent application “Vorrichtung zur Simulation einer Wunde am offenen Abdomen” [Device for simulating a wound on the open abdomen], which was filed at the same time by the applicant of the present invention. Reference is herewith made to the content of this patent application.

The measuring device (50) for simulating a wound on the open abdomen, which is described in the aforementioned patent application and suitable for testing the drainage capacity of the wound dressing, comprises a multiplicity of first hollow bodies (51), filled with a first fluid and having a flexible wall structure, and a second hollow body (52), having a flexible outer enveloping structure, wherein the outer enveloping structure has at least a first opening (53) for simulating a wound. Here the first hollow bodies (51) filled with a first fluid are present in the interior of the second hollow body (52).

FIG. 5 schematically illustrates a complete measuring device (50) for wound bandages with applied NPWT abdominal bandage. The second hollow body (52) has a transparent envelope. A multiplicity of water-filled first hollow bodies (51) were introduced into the second hollow body (52). The outer enveloping structure of the second hollow body (52) has a first opening (53) which simulates the wound, and also a second opening (54) which enables a continuous supply of blood substitute solution into the interior of the second hollow body. The inner side of the envelope of the second hollow body (52) can optionally be coated with an additional ply of a flexible material (521), for example with a foam mat made of polyurethane, which has a thickness of 1 cm. Provision is furthermore made for a fluid source (531) such that blood substitute solution can be routed into the interior of the second hollow body is (52) via the line (532) and via the second opening (54). The fluid source (531) comprises a storage container with blood substitute solution and a pump coupled thereto (not illustrated in FIG. 5). The conduit (532) is routed into the interior of the second hollow body (52) through the second opening (54). Alternatively, the conduit (532) can be connected to a suitable port (not illustrated in FIG. 5), which is provided on the second opening (54).

The measuring device furthermore comprises an NPWT system (negative-pressure therapy apparatus 41) with a negative-pressure source (533), a collection can (534) for receiving the fluid suctioned out of the interior of the second hollow body (52) and a drainage line (535) provided for connection to the negative-pressure port (port, 536) of a negative-pressure therapy bandage. The abdominal bandage illustrated in FIG. 5 comprises a film portion (537), a foam ply (538), a cover film (539) and a negative-pressure port (536). The film portion (537) of the bandage is placed directly on the artificial wound bed (formed by the first hollow bodies (51)) in the manner of a wound contact layer. The film portion (537) is inserted into the interspace formed by abdominal wall (simulated by the second hollow body (52)) and intestines (simulated by the first hollow body (51)). In order to increase the drainage capacity of the bandage, it is possible to apply conduit sections, preferably in a star-shaped arrangement, onto the film portion (537) (not illustrated in FIG. 5). The abdominal bandage furthermore comprises a foam ply (538) made of an open-cell polyurethane foam. Extent and thickness of the foam (538) should be matched as precisely as possible to the artificial wound (first opening 3 in the second hollow body (52)) such that foam edges and wound edges ideally come to rest abutting one another. An airtight self-adhesive cover film (539) made of a polyurethane film is placed over the artificial wound (53) and it is fixed on the surface of the second hollow body (52) outside of the region of the artificial wound. The measuring device (540) serves for establishing the fluid volume suctioned out of the interior of the second hollow body (52). The measuring device (540) is preferably a set of electronic scales. In this case, the fluid volume can be calculated on the basis of differences in weight. The measuring device (540) ideally has a computer connection such that the measurement values can be recorded continuously throughout the simulation experiment. Provision is furthermore optionally made for a third opening (55) in the second hollow body (52). Further measuring instruments, e.g. a pressure gage or a is temperature sensor, can be inserted into the interior of the second hollow body (52) via this opening. Additional pressure sensors can optionally be provided in the NPWT bandage (not illustrated in FIG. 5).

If required, heating means and/or a coolant (not illustrated in FIG. 5) can be routed into the interior of the second hollow body (52) through an optionally present fourth opening (56) in order to set the internal temperature of the wound simulator to a desired value.

The first hollow bodies (51) filled with a first fluid can optionally in their entirety be introduced into an additional sack-like envelope, wherein the additional envelope preferably consists of a flexible film material with a thickness of approximately 0.5 to 1.0 mm (not illustrated in FIG. 5). In the region of the artificial wound (53), the hollow bodies (51) encompassed by an additional envelope in their entirety result in a surface property which, in respect of relief and solidity, is comparable to a real wound in the abdominal region.

### Application of the Wound Dressing

During the application for negative-pressure therapy of large-area wounds in the abdominal region, the wound dressing (10) according to the invention is first of all placed on the wound bed (13). The edge regions of the wound dressing (10) can then be inserted between abdominal wall (14) and wound bed (13) to a depth of between approximately 1 and 30 cm in order to achieve an areal cover of the wound bed (13). Optionally provided pockets (25) which are predominantly open to the center of the wound dressing (10) can simplify the insertion of the edge region of the wound dressing (10) between abdominal wall (14) and wound bed (13) by virtue of the treating medical practitioner inserting a stomach or intestinal spatula or optionally a finger into the pockets and thereupon introducing the edge region of the wound dressing between abdominal wall and the wound bed.

The wound dressing (10) advantageously comprises one or more liquid-permeable layers (11). The one or more liquid-permeable layers can comprise a porous polymer foam. Here, wound healing is greatly promoted if the liquid-permeable layer (11) is matched to the shape of the wound such that the wound edges (15) are in complete contact with the one or more liquid-permeable layers (11).

For the purpose of airtight closure of the wound region, an air-impermeable cover material (16) is placed over the wound. The edges of the cover material (16) are adhesively bonded to the intact skin. Moreover, a negative pressure port (18) is attached in order to establish a functional connection of the wound space to a negative-pressure source (21), e.g. a negative-pressure pump, situated outside of the cover material (16) such that negative pressure can be set in the wound space and liquids can be suctioned out of the wound space. The negative-pressure port (18) is preferably adhesively bonded onto the outer side of the cover material (16) that faces away from the wound, with a suitable opening (17) being cut into the otherwise air-impermeable cover material (16) prior to the adhesive bonding. The negative-pressure therapy is initiated by connecting the negative-pressure port (18) to a negative-pressure source (21) and by applying a constant negative pressure or a time-varying negative pressure for a period of a few minutes up to a number of days.

Here, “constant negative pressure” is understood to mean that the negative pressure is kept substantially constant during the treatment. “Substantially constant” means that there may be minor changes in the negative pressure, e.g. 15% more or less, during the treatment.

A preferred constant negative pressure is in the region from at least 80 mm Hg to at most 250 mm Hg, preferably 125 mm Hg.

Here, “time-varying negative pressure” is understood to mean that the negative pressure is varied in a targeted fashion during the treatment. The targeted variation of the air pressure is understood to mean those variations of the air pressure which are adopted when a first intended value for the negative pressure was achieved after applying the negative-pressure bandage. By contrast, the first increasing phase of the negative pressure, which occurs after the application of the bandage until the first intended value is reached, is not encompassed by the term “time-varying negative pressure”. This analogously applies to the reduction in the air pressure to the ambient air pressure, which is required at the end of the treatment and likewise not encompassed by the term “time-varying negative pressure”.

Accordingly, the present invention describes a method for negative-pressure wound therapy, in particular for a wound in the abdominal region, which method comprises the following steps:

a) applying a wound dressing according to the invention according to one of claims 1 to 20 onto the wound bed

b) sealing the wound using a suitable airtight cover 16,

c) attaching a negative-pressure connection means (18),

d) applying negative pressure for at least 30 minutes and for at most 5 days.

### Exemplary Embodiment

### Measuring the Drainage Capacity of Wound Dressings

The drainage capacity of wound dressings was measured using a measuring device, as described above, for simulating a wound on the open abdomen. The measuring device comprises a ball made of PVC and a multiplicity of hollow bodies made of rubber and filled with water. In the interior of the PVC ball, the hollow bodies filled with water were in their entirety introduced into an additional envelope made of polyethylene. An additional flexible material ply (see 521 in FIG. 5) coating the interior of the PVC ball was not present. The ball has a first opening to simulate an artificial wound and a second opening for continuous supply of a blood substitute solution. The negative-pressure bandage with the wound dressing to be tested was connected to a negative-pressure therapy device. A portion of the blood substitute solution was poured directly into the PVC ball via the first opening before the start of the trial and before the negative-pressure bandage was applied. A further portion of the blood substitute solution was supplied continuously during the trial via the second opening by means of a pump. The amount of blood substitute solution suctioned out by the negative-pressure therapy device during the trial was determined by weighing. Moreover, the amount of blood substitute solution present in the negative-pressure bandage (abdominal wound dressing and additional foam ply) after the trial was determined by weighing.

For the purpose of simulating a wound, an ellipsoidal hole with an open area of approximately 295 cm² was cut into a transparent ball made of PVC with a volume of 15 187 cm³ and a wall strength of 1.6 mm. The ball was rotated such that the artificial wound (first opening) produced thus pointed upward.

Approximately 268 ml water was filled in each case into approximately 50 hollow bodies made of rubber with a wall strength of 0.3 mm. The hollow bodies were sealed in a fluid-tight fashion.

A vacuum bag made of polyethylene with a wall strength of approximately 1 mm was introduced into the interior of the PVC ball via the ellipsoidal first opening of the ball. The vacuum bag was dimensioned such that the whole internal space of the PVC ball could be coated therewith. The water-filled hollow bodies made of rubber were, in their entirety, placed into the vacuum bag situated in the interior of the PVC ball via the first opening of the PVC ball, i.e. the hollow bodies made of rubber were situated both in the interior of the PVC ball and in the interior of the vacuum bag as additional envelope. The hollow bodies made of rubber, situated in the vacuum bag and filled with water, filled out the internal cavity of the PVC ball to the greatest possible extent. The vacuum bag filled with hollow bodies made of rubber was closed by folding over the edge thereof.

In order to produce one liter of blood substitute solution, 566 g demineralized water, 425 g glycerin, 9 g NaCl and 0.2 g of the dye Allura Red were mixed. The solution has a viscosity of approximately 4.5 mPa*s at 23° C.

Before the start of the trial and before applying the negative-pressure bandage, 250 ml of blood substitute solution (pre-filling volume) were poured directly into the PVC ball via the first opening.

The abdominal wound dressings to be tested were applied into the artificial wound as follows:

Trial 1 with wound dressing 1: Wound dressing ABThera™ from the producer KCl (USA). The main element of the ABThera™ wound dressing is a slit polyurethane film. Segmented foam strips are applied to the film in star-shaped arrangement. A further ply of a slit film is situated on the side of the arrangement made of film and foam strips that faces away from the wound during use. A further foam ply made of polyurethane (VivanoMed® foam by Paul Hartmann AG, Heidenheim, Germany) was placed onto the central region of the wound dressing such that the edges of the further foam ply are in direct contact with, i.e. butting on, the edges of the artificial wound (first opening cut into the PVC ball). The artificial wound was covered by an airtight cover film, with the cover film being adhesively bonded to the outer surface of the PVC ball in the surroundings of the artificial wound. Negative-pressure communication between the wound space of the artificial wound and a negative-pressure source ATMOS S041 (Atmos, Germany) was established via a negative-pressure port (port).

Trial 2 with wound dressing 2: Prototype for an abdominal wound dressing according to the invention. 6 flat drainage tubes (Primed, Germany) were applied to a perforated film made of polyolefin (Medifol® 3D by rkw ProLife, Wasserburg, Germany) with an overall round shape. The flat drainage tubes were attached to the film by adhesive bonding using an adhesive based on cyanoacrylate. The circular film has a diameter of 60 cm. Each of the conduit sections (section of a flat drainage tube) has a length of 27 cm, a width (B) of approximately 2 cm and a thickness (H) of approximately 0.53 cm. Each of the conduit sections has two continuous cavities which are not interconnected. The cross section of the conduit section is shown schematically in FIG. 6. The Shore A hardness of the conduit section is 60. On the inner side, the conduit section has bulges or thickenings which serve to stabilize the conduit section in negative pressure. At the thinnest point, the wall strength of the conduit section is 0.7 mm. With the exception of the openings situated at the ends, each of the conduit sections has no further openings. The conduit sections are is applied to the perforated film in a star-shaped arrangement, with the conduit sections extending up to the edge of the film. The ends of the conduit sections oriented toward the center of the wound dressing are spaced apart from one another. The spacing a (see FIG. 1 a, section) between the ends of two conduit sections opposite to one another is approximately 6 cm.

The sum of the open area of the openings (6) present in the first film (1) is 14% of the areal extent of the film. The diameter of the openings (6) present in the first film (1) is 0.3 mm, with there being approximately 168 openings (6) per cm². The film has a mass per unit area of 60 g/m², with the mass per unit area being determined pursuant to the EN ISO 2286-2 norm. The tensile stress of the film, determined pursuant to EN ISO 527, is (MD/CD) 22/17 N/inch. The tensile strain at break of the film, determined pursuant to EN ISO 527, is (MD/CD) 500/500%. The polyolefin film has funnel-shaped or conically-shaped openings which have a three-dimensional structure such that, as a result of this, the film has a smooth side and a roughened side situated opposite to the smooth side. The smooth side is provided for application on the artificial wound bed while the conduit sections are attached to the roughened side.

As described in the case of wound dressing 1, a further foam ply made of polyurethane (VivanoMed-®foam by Paul Hartmann AG, Heidenheim, Germany) was applied to the central region of the wound dressing such that the edges of the further foam ply are in direct contact with, i.e. butting on, the edges of the artificial wound (opening cut into the PVC ball). The airtight cover of the artificial wound and the connection to a negative-pressure source was brought about as described above in respect of wound dressing 1.

The trial duration was 60 min in each case. A constant negative pressure of 125 mm Hg was applied.

Over the trial period of 60 min, a total of approximately 500 ml of a colored blood substitute solution was introduced continuously and with unchanging flow rate (exudate inflow) via a line connected to the second opening (see 54 in FIG. 5) of is the ball.

The trial was carried out at room temperature (23° C.). There was no heating of the blood substitute solution, i.e. the blood substitute solution likewise had a temperature of approximately 23° C. The blood substitute solution suctioned out by the NPWT system was collected in the can of the negative-pressure unit. The volume of the blood substitute solution suctioned into the can after 60 min was calculated on the basis of the difference in weight between empty can (before the start of the trial) and filled can (after carrying out the trial). Furthermore, the volume of the blood substitute solution present in the negative-pressure bandage (abdominal wound dressing and additional foam ply) after the trial was calculated on the basis of the difference in weight between dry negative-pressure bandage (before the start of the trial) and moist negative-pressure bandage (after carrying out the trial). The volume of the blood substitute solution remaining in the wound simulator (PVC ball filled with hollow bodies made of rubber) was calculated:

| | Wound- | | Wound | | |
|---|---|---|---|---|---|
| | simulator | Can | dressing | Foam | Sum |
| | (ml) | (ml) | (ml) | (ml) | (ml) |
| Trial 1 | 194.0 | 276.6 | 163.5 | 115.9 | 750.0 |
| Trial 2 | 152.8 | 455.2 | 25.5 | 116.5 | 750.0 |

Overall, a greater volume of blood substitute solution (455.2 ml compared to 276.6 ml) was suctioned into the negative-pressure source in the experiment (trial 2) carried out with wound dressing 2 than in the experiment (trial 1) carried out with wound dressings 1. It follows that, under the aforementioned trial conditions, wound dressing 2 exhibits a comparatively higher drainage capacity at the wound simulator.

A greater volume of blood substitute solution was retained in the wound dressing during trial 1.

On the basis of the coloring of the blood substitute solution, it was possible to observe during the trial that the blood substitute solution barely penetrates the interspace formed by the foam ply and film ply in both wound dressings 1 and 2. Instead, the blood substitute solution appears preferably to flow between the artificial is intestines (surface of the hollow bodies made of rubber and surrounded by an additional envelope) and the wound-side surface of the slit film to the center of the wound dressing and to be suctioned out at said point. The increased drainage capacity of the wound dressing 2 according to the invention compared to wound dressing 1 could be due to an improved outflow of blood substitute solution present in the peripheral region of the artificial wound through the open lumina of the conduit sections.

What is claimed is:

1. A wound dressing for use in negative-pressure wound therapy, the treatment of abdominal wounds, comprising
i) a first flexible film (1) with a first and a second side, with the first side being provided for application on the wound bed (13), more particularly on exposed internal organs or on the greater omentum, and with the first film (1) having a multiplicity of openings (6) distributed across the area,
ii) at least three conduit sections (2), applied to the second side of the first film (1), made of a flexible elastomeric material with a thickness (H) of at most 20 mm, with each of the conduit sections (2) having at least one continuous cavity (3), and with each of the conduit sections (2) having a flat design, characterized in that, with the exception of the openings (5) situated at the ends, each of the conduit sections (2) has no further openings.

2. The wound dressing according to claim 1, wherein the conduit sections (2) are connected in non-detachable and areal fashion to the second side of the first film (1) with at least 25% of their area projected perpendicularly onto the first film (1), preferably with at least 50% of their area projected perpendicularly onto the first film (1).

3. The wound dressing according to claim 1, wherein the conduit sections (2) are made of a material which has a Shore A hardness of at least 20 and of at most 70, preferably a Shore A hardness of at least 30 and of at most 65, more particularly a Shore A hardness of at least 40 and of at most 62, and wherein the drainage tube can furthermore comprise EVA, PU, PVC, PE, PET, PTFE, TPE, silicone or a mixture thereof.

4. The wound dressing according to claim 1, wherein the conduit sections (2) are sections of a single-lumen or multi-lumen drainage tube and wherein the conduit sections (2) can be produced by cutting a longer drainage tube to length.

5. The wound dressing according to claim 1, wherein at least three conduit sections (2) are applied to the second side of the first film (1) and wherein the at least three conduit sections (2) are applied to the first film (1) in a cross-shaped or star-shaped arrangement.

6. The wound dressing according to claim 1, wherein the sum of the open area of the openings (6) present in the first film (1) is at least 0.1% and at most 25% of the areal extent, preferably at least 10% and at most 18% of the areal extent, more particularly at least 13% and at most 15% of the areal extent of the film.

7. The wound dressing according to claim 1, wherein the diameter of the openings (6) present in the first film (1) is at least 0.2 mm and at most 0.4 mm, more particularly 0.3 mm.

8. The wound dressing according to claim 1, wherein the number of openings (6) present per unit area in the first film (1) is at least 150 per cm² and at most 300 per cm².

9. The wound dressing according to claim 1, wherein the mass per unit area of the first film (1) is at least 30 g/m² and at most 150 g/m², preferably at least 45 g/m² and at most 95 g/m² and more particularly at least 55 g/m² and at most 65 g/m².

10. The wound dressing according to claim 1, wherein the openings (6) present in the first film (1) have a conical or cylindrical three-dimensional shape to the greatest possible extent and, as a result of this, the film has a smooth side and a roughened side which lies opposite to the smooth side.

11. The wound dressing according to claim 10, wherein the first side of the first film (1) provided for application on the wound bed (13) is formed by the smooth side of the first film (1) and the second side of the first film (1) is formed by the roughened side.

12. The wound dressing according to claim 1, wherein the conduit sections on the second side of the first film (1) are present separated from one another such that there is no formation of a continuous cavity that connects the present conduit sections in a fluid-conducting fashion.

13. The wound dressing according to claim 1, furthermore comprising a second flexible film (4), wherein the second film (4) is applied in a non-detachable fashion to the side of the composite of first film (1) and conduit sections (2) that faces away from the wound during use.

14. The wound dressing according to claim 13, wherein the second film (4) has a multiplicity of openings distributed across the area thereof, which openings are suitable for passing fluids through them, or wherein the second film is embodied to is be impermeable to fluid to the greatest possible extent and merely has a single opening (10), the latter preferably being arranged in the central region.

15. The wound dressing according to claim 1, wherein the first flexible film (1) and/or—if present—the second flexible film (4) is a thermoplastic film made of EVA, PU, PE, PET, PTFE, PVC, TPE, silicone or a mixture thereof.

16. The wound dressing according to claim 1, furthermore comprising a connection element (8), which can be applied to the second side of the first flexible film (1) and to which the end sections of the at least three conduit sections (2) can be attached in a non-detachable fashion.

17. The wound dressing according to claim 1, wherein the wound dressing has at least one pocket (25) which is open toward the center of the wound dressing on the side that faces away from the wound during use and which simplifies the uniform application of the wound dressing to the wound bed (13) and the laying out of said wound dressing on the latter, wherein the pocket (25) is preferably formed by the application of further pieces of material, more particularly pieces of film.

18. The wound dressing according to claim 1, furthermore comprising one or more liquid-permeable layers (11), for application on the side of the wound dressing that faces away from the wound during use.

19. The wound dressing according to claim 18, wherein the liquid-permeable layer (11) comprises a porous polymer foam, fabricated from PU, PVA or silicone.

20. A bandage for use in negative-pressure wound therapy in the treatment of abdominal wounds, comprising a wound dressing according to one of preceding claim 1, furthermore comprising an air-impermeable cover material (16) for airtight sealing of the wound and the surroundings of the wound, wherein the cover material (16) preferably has an adhesive edge.

21. A device for use in negative-pressure wound therapy for treatment of abdominal wounds, comprising a bandage according to claim 20, and furthermore comprising a negative-pressure port, which can be applied to the air-impermeable cover material (16) on a side that faces away from the wound, for the functional connection of the wound space to a negative-pressure source (21) situated outside of the cover material (16) such that negative pressure can be set in the wound space and liquids can be suctioned out of the wound space.

22. A kit for negative-pressure wound therapy for treatment of abdominal wounds, comprising
a) a wound dressing according to claim 1,
b) at least one liquid-permeable layer (11) for application on a side of the wound dressing that faces away from the wound during use, wherein the at least one liquid-permeable layer (11) comprises one or more areal cushions of a porous polymer foam of PU, PVA or silicone,
c) an air-impermeable cover material (16) for airtight closure of the wound and the surroundings of the wound, wherein the cover material (16) preferably has an adhesive edge,
d) a negative-pressure connection means (18) for the functional connection of the wound space to a negative-pressure source (21) situated outside of the cover material such that negative pressure can be set in the wound space and liquids can be suctioned out of the wound space, wherein the negative-pressure connection means (18) is provided for application to an outer side of the cover material that faces away from the wound during use,
and wherein components a) to d) are present in sterile packaged form.

23. Method for producing a wound dressing according to claim 1, comprising the following steps:
a) providing of a first flexible film (1) with a first and a second side, with is the first side being provided for application on the wound bed (13), more particularly on exposed internal organs or on the greater omentum, and with the film (1) having a multiplicity of openings (6) distributed across the area;
b) providing of at least three conduit sections (2) made of a flexible elastomeric material with a thickness (H) of at most 20 mm, with each of the conduit sections (2) having at least one continuous cavity (3), and with each of the conduit sections (2) having a flat design, and wherein furthermore, with the exception of the two openings (5) situated at the ends, each of the conduit sections (2) has no further openings;
c) optionally providing a connection element (8), to which the end sections of the at least three conduit sections (2) can be attached in a non-detachable fashion and which can be applied to the second side of the first flexible film (1), preferably in a central fashion;
d) optionally connecting end sections of the at least three conduit sections (2) to the connection element (8);
e) applying the at least three conduit sections (2) or the composite of the conduit sections and the connection element (8) to the second side of the first film (1), in a cross-shaped or star-shaped arrangement of the conduit sections (2) on the first film (1); and
f) optionally applying a second flexible film (4) onto the composite of first film (1) and conduit sections (2), wherein the second film is applied to a side of the composite that points away from the wound during use.
